(19) United States
(12) Patent Application Publication
(10) Pub. No.: US 2003/0026840 A1
(43) Pub. Date: Feb. 6, 2003

(54) Combinations for introducing nucleic acids into cells

(76) Inventors: Christian Plank, Seefeld (DE); Axel Stemberger, Neubiberg (DE); Franz Scherer, Lenggries (DE)

Correspondence Address: FISH & NEAVE, 1251 AVENUE OF THE AMERICAS, 50TH FLOOR, NEW YORK, NY, 10020-1105, US

(21) Appl. No.: 10/023,317

(22) Filed: Dec. 17, 2001

Related U.S. Application Data
(63) Continuation of application No. PCT/EP00/05778, filed on Jun. 21, 2000.

(30) Foreign Application Priority Data
Jun. 25, 1999 (EP) EP 99 11 2260.7
Nov. 24, 1999 (DE) DE 199 56 502.3

(51) Int. Cl. A61K 48/00; C07K 5/00; A61K 9/14

(52) U.S. Cl. 424/486; 514/44; 530/324

(57) ABSTRACT

Combinations of a carrier and a complex consisting of a nucleic acid molecule and a copolymer are described, wherein the copolymer consists of an amphiphilic polymer, preferably polyethylene glycol, and a charged effector molecule, in particular a peptide or peptide derivative, as well as their use for the transfer of nucleic acid molecules into cells.

The invention relates to the field of gene transfer, in particular to combinations of a carrier and a complex consisting of a nucleic acid molecule and a copolymer.

A prerequisite for putting strategies of gene therapy clinically into practice is the availability of stable, efficient gene vectors. In the systemic application aimed at the somatic gene therapy, most of the known gene transfer vehicles, however, still incur problems.

In principle, the two following transport problems are to be solved to achieve an efficient gene transfer in vivo: 1) transfer of the agent to be transferred (e.g. plasmid DNA, oligonucleotide) from the application site in the organism to the target cell (extra-cellular aspect) and 2) transfer of the agent to be transferred from the cell surface into the cytoplasm or the nucleus (cellular aspect). An essential precondition for the gene transfer mediated by receptors is to compact the DNA to particles having the size of a virus and to release the DNA from internal vesicles after the endocytotic intake in the cells. This precondition is fulfilled by compacting the DNA with specific cationic polymers the chemical nature of which guarantees the release of DNA complexes from internal vesicles (endosomes, lysosomes) after the endocytotic intake in the cells (Boussif et al., 1995; Ferrari et al., 1997; Haensler & Szoka, 1993; Tang et al., 1996). Such an effect is also achieved by incorporating pH-dependent membrane-destroying peptides into DNA complexes (Plank et al., 1994; WO 93/07283). Using a suitable composition of the DNA complexes, a specific intake and an efficient gene transfer into the cells can be achieved by means of receptor-ligand interaction (Kircheis et al., 1997; Zanta et al., 1997). Complexes of DNA with cationic peptides are also particularly suitable for the gene transfer mediated by receptors (Gottschalk et al., 1996; Wadhwa et al., 1997; Plank et al., 1999).

Amongst others, the fact that the extra-cellular aspect of the transport problem has only been solved insufficiently renders it more difficult to put the promising research findings which can be achieved with non-viral vectors clinically into practice. One reason for this problem is the physicochemical nature of the non-viral gene transfer vectors due to which they strongly interact with blood and tissue components during the systemic application (e.g. by opsonization, the attachment of serum protein) which particularly limits the receptor-mediated gene transfer directed to certain target cells. It was shown that the modification of the surface of DNA complexes with poly(ethylene glycol) considerably reduces their blood protein-binding characteristics (Plank et al., 1996; Ogris, 1998; WO 98/59064). Another limitation of the use of non-viral vectors is the insufficient solubility (or stability) of DNA complexes in vivo. With the known methods it has not been possible so far to complex DNA with a polycation for intravenous application in concentrations sufficiently large (e.g. in the range of 1 mg/ml) since the DNA complexes aggregate under physiological saline concentrations and precipitate from the solution.

Similar problems also occur during the application of low-molecular chemical compounds. In the field of “classic” medicaments, biologically degradable synthetic polymers are used for packaging pharmaceuticals in a form that guarantees a longer retention time in the organism and that leads to the desired biological availability in the target organ (“controlled release”). For this purpose, the modification of the surface of colloidal particles with polyethylene glycol is formed in such a way that the undesired opsonization is suppressed. There is extensive literature on the synthesis and characterisation of biologically degradable polymers for use in numerous medical applications (Coombes et al., 1997). Depending on the substance and the application, the chemical bindings in the backbone of the polymer are varied. The desired lability in a physiological milieu can be achieved by means of the suitable positioning of ester, amide, peptide or urethane bonds, by which the sensitivity to the action of enzymes can be varied purposefully. Combinatorial synthesis principles have proven to be effective for a fast and efficient synthesis of biologically effective substances (Balklenhohl et al., 1996). By systematically varying only few parameters, a large number of compounds can be obtained which have the desired basic structure (Brocchini et al., 1997). Using a suitable, meaningful biological selection system, it is possible to select from this pool of compounds the ones which have the desired characteristics.

In the U.S. Pat. No. 5,455,027, polymers are described which consist of alternating units of a polyalkylene oxide and a fuctionalised alkane, wherein a pharmacologically active agent is covalently coupled to the functional side group of the alkane.

In the course of the recent years, the following essential points have become apparent as regards the application of non-viral gene transfer systems:

a) Complexes of plasmid DNA and cationic polymers are suitable for a gene transfer in vitro and in vivo, wherein complexes with polymers having secondary and tertiary amino groups can also have an inherent endosomolytic activity leading to an efficient gene transfer (Boussif et al., 1995, Tang et al., 1996).

b) From a certain chain length of the cationic portion, branched cationic peptides are suitable for efficiently binding to DNA and for forming particular DNA complexes (Plank et al., 1999).

c) Polycation DNA complexes strongly interact with blood components and activate the complement system (Plank et al., 1996).

d) Strong interactions of particulate structures with blood components can be reduced or inhibited by modification with polyethylene glycol; this also applies to polycation DNA complexes (Plank et al., 1996; Ogris et al., 1999).

Therefore, the technical problem underlying the present invention was to provide a new, improved non-viral gene transfer system on the basis of nucleic acid-polycation complexes.

For solving the technical problem underlying the present invention, it was assumed that nucleic acid or nucleic acid complexes are to be coated with a charged polymer which physically stabilises the complexes and protects them from opsonization.

The present invention relates in its first aspect to a charged copolymer having the general formula I

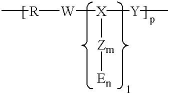

wherein R is an amphiphilic polymer or a homo- or hetero-bifunctional derivative thereof,

and wherein X

i) is an amino acid or an amino acid derivative, a peptide or a peptide derivative or a spermine or a spermidine derivative; or

ii) wherein X is

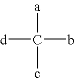

wherein

a is H or, optionally halogen- or dialkylamino-substituted, C₁-C₆ alkyl;

and wherein

b, c and d are the same or different, optionally halogen- or dialkylamino-substituted, C₁-C₆ alkylene; or

iii) wherein X is

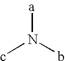

wherein

a is H or, optionally halogen- or dialkylamino-substituted, C₁-C₆ alkyl,

and wherein

b and c are the same or different, optionally halogen- or dialkylamino-substituted, C₁-C₆ alkylene; or

iv) wherein X

is a substituted aromatic compound with three functional groupings W₁Y₁Z₁, wherein W, Y and Z have the meanings mentioned below;

wherein

W, Y or Z have the same or different groups CO, NH, O or S or a linker grouping capable of reacting with SH, OH, NH or NH₂;

and wherein the effector molecule E is a cationic or anionic peptide or peptide derivative or a spermine or spermidine derivative or a glycosaminoglycane or a non-peptidic oligo/polycation or -anion;

wherein

m and n are independently of each other 0, 1 or 2; wherein

p preferably is 3 to 20; and wherein

I is 1 to 5, preferably 1.

If I is >1, the moiety X—Zₘ—Eₙ is the same or different.

Within the meaning of the present invention, an aromatic compound is a monocyclic or bicyclic aromatic hydrocarbon group with 6 to 10 ring atoms which—apart from the aforementioned substituents—can optionally be independently substituted with one or more further substituents, preferably with one, two or three substituents selected from the group of C₁-C₆-alkyl, —O—(C₁-C₆-alkyl), halogen—preferably fluorine, chlorine or bromine—cyano, nitro, amino, mono-(C₁-C₆-alkyl)amino, di-(C₁-C₆-alkyl)amino. The phenyl group is preferred.

Within the meaning of the present invention, an aromatic compound can also be a heteroaryl group, i.e.: a monocyclic or bicyclic aromatic hydrocarbon group with 5 to 10 ring atoms which contains independently of each other one, two or three ring atoms selected from the group of N, O or S, wherein the remaining ring atoms are C.

Unless stated otherwise, alkylamino or dialkylamino is an amino group which is substituted with one or two C₁ to C₆ alkyl groups, wherein—in the case of two alkyl groups—the two alkyl groups may also form a ring. Unless stated otherwise, C₁ to C₆ alkyl generally represents a branched or unbranched hydrocarbon group with 1 to 6 carbon atom(s) which can optionally be substitued with one or more halogen atom(s)13 preferably with fluorine—which may be different from each other or the same. Examples thereof may be the following hydrocarbon groups: methyl, ethyl, propyl, 1-methylethyl (isopropyl), n-butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Unless stated otherwise, low alkyl groups having 1 to 4 carbon atoms, such as methyl, ethyl, propyl, iso-propyl, n-butyl, 1-methylpropyl, 2-methylpropyl or 1,1-dimethylethyl are preferred.

Accordingly, alkylene means a branched or unbranched divalent hydrocarbon bridge having 1 to 6 carbon atoms which may optionally be substituted with one or more halogen atom(s)—preferably fluorine—which may be different from each other or the same.

The amphiphilic polymer R is preferred to be a polyaklylene oxide, polyvinyl pyrollidone, polyacryl amide, polyvinyl alcohol or a copolymer of these polymers.

Examples of suitable polyalkylene oxides are polyethylene glycols (PEG), polypropylene glycols, polyisopropylene glycols, polybutylene glycols.

Within the framework of the present invention, polyalkylene oxides, in particular PEG, are preferred.

The polyalkylene oxide may be present as such in a copolymer or as thio-, carboxy- or amino derivative.

The polymer R preferably has a molecular weight of 500 to 10,000, preferably 1,000 to 10,000.

In the case i) in which X is an amino acid, an amino acid with three functional groups can be used for the synthesis of the copolymer, wherein two of these groups are capable of copolymerisation with the polymer and one of coupling with the effector molecule E; in this case, Z is not necessary. The natural amino acids glutamic acid, aspartic acid, lysine, ornithine and tyrosine are preferred. In principle, synthetic amino acids may also be used instead of natural amino acids (e.g. corresponding spermine and spermidine derivatives).

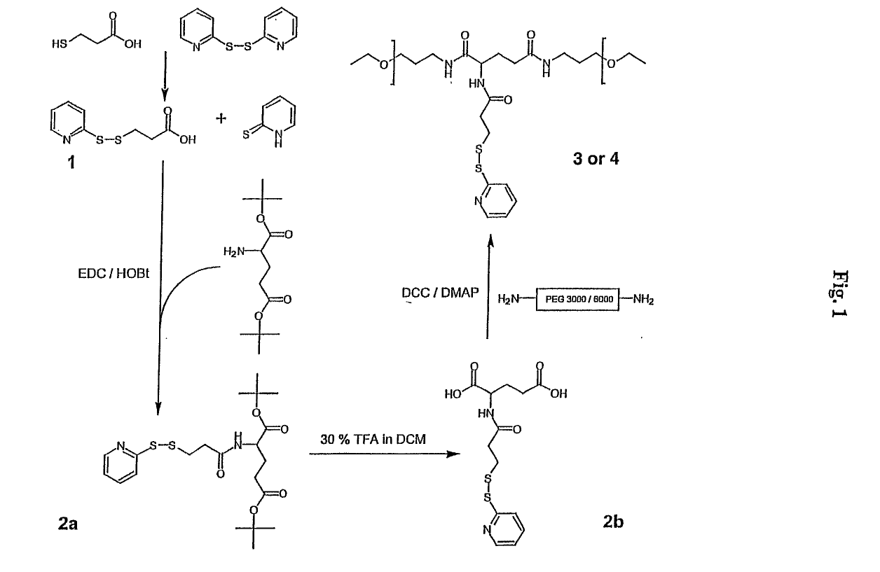

In the case i) an amino acid derivative may also be used for the synthesis, the amino acid derivative having two functional groups for the copolymerisation with the polymer and being obtained by modification of an amino acid (glutamic acid, aspartic acid, lysine or ornithine) with a linker grouping for coupling with the effector molecule. Thus, Z is not necessary (m=0); examples of linker groupings are pyridylthiomercaptoalkyl carboxylates (cf. FIG. 1) or maleimidoalkane carboxylates.

In the case i) X may also be a peptide (derivative). If the peptide or the peptide derivative is not charged, E is coupled thereto directly or via Z.

If X is a positively or negatively charged peptide or peptide derivative or a spermine or spermidine derivative, X itself represents the effector molecule (Z and E are not necessary, m=n=0). In the simplest case, the peptide consists in this case of a linear sequence of two or more identical or different natural or synthetic amino acids, wherein the amino acids are selected in such a way that the peptide is altogether either negatively or positively charged. Alternatively, the peptide may also be branched. In these cases, the peptide as such is the effector, Z and E are not necessary (m=n=0). Examples of a type of suitable cationic peptides have been described by Plank et al., 1999.

Suitable anionic peptide derivatives X have the general formula (peptide)ₙ-B-spacer-(Xaa). The peptide is a sequence of amino acids or amino acid derivatives with a negative charge altogether. Preferably, the peptide consists of three to 30 amino acids, more preferably, it consists only of glutamic acid and/or aspartic acid residues. n represents the number of branchings depending on the functional groups contained in B. B is a branching molecule, preferably lysine or a molecule of the type X in the cases ii) to iv). The spacer is a peptide consisting of 2 to 10 amino acids or an organic amino carboxylic acid having 3 to 9 carbon atoms in the carboxylic acid backbone, e.g. 6-aminohexane acid. The spacer serves the spatial separation of the charged effector molecule from the polymer backbone. Xaa preferably is a trifunctional amino acid, in particular glutamic acid or aspartic acid and can generally be a compound of the type X, in the cases i) to iv).

Alternatively, in the case i) X can be a peptide derivative, wherein the modification of the peptide is a charged grouping which is different from an amino acid; examples of such groupings are sulfonic acid groupings or charged carbohydrate groups such as neuraminic acids or sulfated glycosaminoglycans. The modification of the peptide can be carried out according to standard methods, either directly in the course of the peptide synthesis or afterwards with the finished peptide.

As in case i), the effector molecule E can be a polycationic or polyanionic peptide or peptide derivative or a spermine or spermidine derivative. In the simplest case, the peptide is also in this case a linear sequence of two or more identical or different natural or synthetic amino acids, wherein the amino acids are selected in such a way that the peptide altogether is charged either positively or negatively. Alternatively, the peptide can be branched. Examples of suitable branched cationic peptides have been described by Plank et al., 1999. Suitable anionic molecules E have the general formula (peptide)ₙ-B-spacer-(Xbb), wherein Xbb preferably is an amino acid with a reactive group which can be coupled to X directly or via Z.

The coupling of the effector peptide E to Z or directly to X is carried out via a reactive group which either exists in the peptide from the beginning or which is introduced afterwards, e.g. a thiol group (in a cysteine or by introducing a mercaptoalkane acid group). Alternatively, depending on Z, the coupling may also take place via existing amino or carboxylic acid groups or via amino or carboxylic acid groups introduced afterwards.

As in case i), E can alternatively be a peptide derivative, wherein the modification of the peptide is a charged grouping which is different from an amino acid, examples of such groupings are sulfonic acid groupings or charged carbohydrate groups such as neuraminic acids or sulfated carbohydrate groups. In this case, too, the coupling to X takes place directly or via Z.

The copolymer of the general formula I

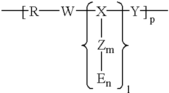

is preferred to be structured as a strongly alternating block copolymer.

Optionally, the copolymer is modified with a cellular ligand for the target cell (receptor ligand L). In this case, in most of the linker positions Z there is an E. Between them, instead of the cationic or anionic effector E, a cellular ligand is coupled to individual positions of the linker Z. Alternatively, the ligand is coupled to individual positions of the effector molecule E. Preferably, the ratio of E to L is approximately 10:1 to 4:1.

The receptor ligand may be of biological origin (e.g. transferrin, antibodies, carbohydrate groups) or synthetic (e.g. RGD-peptides, synthetic peptides, derivatives of synthetic peptides); examples of suitable ligands are indicated in WO 93/07283. The copolymers of the invention, can be produced according to the following method:

If it is a peptide or peptide analogue, the copolymerisation partner X or X-Zₘ-Eₘ is synthesised according to standard methods following the Fmoc protocol (Fields et al., 1990), e.g. at the solid phase (solid phase peptide synthesis, SPPS). The amino acid derivatives are activated with TBTU/HOBt or with HBTU/HOBt (Fields et al., 1991). For the ionic amino acid positions, the following derivatives are used in their N-terminal Fmoc-protected form:

(a) cationic side chains: R(Pbf), K(Boc, Trt), ornithine (Boc), carboxy spermine or—spermidine (Boc).

(b) anionic side chains; D(O-tert. Bu), E(O-tert. Bu).

For the branching site B of the molecule (peptide)ₙ-B-spacer-(Xaa) or (peptide)ₙ-B-spacer-(Xbb), Fmoc-K-(Fmoc)-OH is used. The peptides are separated from the resin with TFA/DCM.

If the polymerisation partner X is a peptide having the general structure (peptide)n-B-spacer-(Xaa) in the subsequent copolymerisation, glutamic acid or aspartic acid which has a benzyl protecting group at a carboxyl position is used at the position Xaa. This is selectively removed by hydrogenolysis (Felix et al., 1978). The N-terminal amino acid positions of the peptide chain have Boc-protected amino acids so that the protecting groups can be separated in one step after copolymerisation of the peptide with PEG.

If the polymerisation partner X is an amino acid derivative which contains a linker grouping (e.g. 3-mercaptopropionic acid, 6-aminohexane acid), it can be obtained in liquid phase according to classic methods of peptide chemistry . Mercaptopropionic acid is reacted with 2,2′-dithiodipyridine and purified chromatographically. The reaction product is reacted with carboxyl-protected glutamic acid (O-t.butyl) using HOBt/EDC activation (cf. FIG. 1). 6-Fmoc-aminohexane acid is reacted analogously. The carboxyl protecting groups are removed in TFA/DCM, the resulting glutamic acid derivative is purified using chromatographic methods.

The production of the copolymers can be effected according to the following principles and is illustrated by way of a PEG-peptide copolymer:

(1) Poly(PEG-O-OC-) Matrix (“Polyester”)

The copolymerisation of the ionic, partially side-chain-protected peptide-dicarboxylic acids or glutamic or aspartic acid derivatives with PEG-macromonomers in defined molecular mass ranges (MW 400-20,000 commercially available, e.g. Fluka) results in a matrix on a PEG-ester basis. This is a system which is hydrolysis-labile in a physiological milieu (Ulbrich et al., 1985).

The p(PEG-peptide)-copolymers are formed according to established methods, e.g. with dicyclohexylcarbodiimide/DMAP, preferably in a strongly alternating sequence (Zalipsky et al., 1984; Nathan, A., 1992). To the PEG-macromonomer present together with a side-chain-protected peptide or glutamic or aspartic acid derivative in a dichloromethane solution, DCC/DMAP is added. After separating the resulting urea derivative, the polymer can be obtained by means of precipitation with cold ether. The remaining side chain protecting groups are separated with TFA in dichloromethane (under these conditions, the PEG-ester binding is stable, too (Zalipsky et al., 1984)). The ionic polymer is obtained by precipitation and a final chromatographic step. Reaction engineering allows to control the polymerisation degree and the ratio of charge per PEG unit in the polymer.

(2) Poly(PEG-HN-OC) Matrix (“Polyamide”)

As an alternative to the polyester, an amidic polymer matrix may be constructed if the capability of hydrolysis is expected to be too fast and thus the instability is expected to be too high in the case of systemic application, when the copolymer-DNA complex is used in a gene therapeutic application. In this case, instead of the PEG macromonomers, diamino-PEG derivatives are used which are copolymerised with the ionic peptides or the glutamic or aspartic acid derivatives analogously to the above-described synthesis. During this synthesis, a hydrolysis-stable amide structure is obtained. Diamino-modified polyethylene glycols are commercially available as basic substances in defined molecular mass ranges between 500 and 20,000 (e.g. Fluka). The remaining acid-labile side-chain protecting groups of the peptide components are separated, e.g. with TFA/DCM, and the polymers are purified by means of chromatographic methods.

In another step, copolymers of glutamic or aspartic acid derivatives are reacted with anionic or cationic peptides which contain a suitable reactive group. Copolymers of the 3-(2′-thio-pyridyl)-mercaptopropionyl-glutamic acid, for instance, are reacted with peptides which contain a free cysteine-thiol group. From copolymers resulting from 6-Fmoc-aminohexanoyl-glutamic acid, the Fmoc group is removed under alkaline conditions. The product is reacted with a carboxyl-activated, protected peptide. The peptide protecting groups (t-Boc or O-t. butyl) are removed in DCM/TFA, the resulting product is purified chromatographically. Alternatively, the amino group of Ahx can be derivatised with bifunctional linkers and then reacted with a peptide.

The ligand L can be coupled directly by activating carboxyl groups at the effector E (preferably in the case of anionic copolymers) or at the ligand or by inserting bifunctional linkers such as succinimidyl-pyridyl-dithioproprionate (SPDP; Pierce or Sigma) and similar compounds. The reaction product can be purified by gel filtration and ion exchange chromatography.

This copolymerisation mixture can also be reacted according to combinatorial principles. In this case, mainly the type and the molecular weight (polymerisation degree) of the polymer R, the identity of the polymerisation partner X-Zₘ-Eₙ or the effector molecule E (e.g. a series of anionic peptides with an increasing number of glutamic acids) and the total polymerisation degree p are the selectable variable.

By varying the molecular masses of the PEG macromonomers, the kind of the ionic species used as well as their share in the copolymer and the polymerisation degree of the polymer matrix, a system of several parameters is established which allows for the fast parallel construction of a homologous sequence of different copolymers and, subsequently, after complexing with the nucleic acid, of various non-viral vectors. The synthesis concept is put into practice on the scale of a cell culture plate (e.g. 96 wells per plate). For this purpose, the chemical synthesis is adapted to the required micro scale (reaction volumes in the range of 500 μl). This allows for the direct transfer of the polymers synthesised simultaneously in the biologic assay and thus contributes to a fast screening of a plurality of systems and for the identification of suitable compounds. For carrying out the biological selection method with regard to the preferred use of the copolymers of the invention for the gene transfer, the copolymers are reacted with, for instance, DNA complexes and are then subjected to tests which permit an assessment of the features of the polymer as to the intended use (e.g. gene transfer). Such selection methods can be used for nanoparticles coated with copolymers. Such screening and selection methods can, for instance, serve complement activation tests in a 96-well-plate format (Plank et al., 1996), or be turbidimetric measurements of the aggregation induced by serum albumin or salt in the same format or in-vitro gene transfer studies in the same format (Plank et al., 1999) or fluorescence-optical methods in the same format.

Such analyses show, for example, which copolymers of a combinatorial synthetic mixture are suitable for modifying the surface of DNA complexes in such a way that their solubility is sufficient for gene transfer applications in vivo, their interaction with blood and tissue components is reduced so that their time of retention and the duration of effect in the blood circulation is sufficiently increased for the receptor-mediated gene transfer into the target cells to take place.

The copolymers of the invention are preferably used for the transport of nucleic acids into higher eukaryotic cells.

Therefore, in another aspect the present invention relates to complexes containing one or more nucleic acid molecules and one or more charged copolymers of the general formula I.

Preferably, the nucleic acid molecule is condensed with an organic polycation or a cationic lipid.

In another aspect, the invention thus relates to complexes of nucleic acid and an organic polycation or a cationic lipid which are characterised in that they have a charged copolymer of the general formula I bound to their surface via ionic interactions.

The nucleic acids that are to be transported into the cell can be DNAs or RNAs, wherein there are no restrictions as to the nucleotide sequence and the size. The nucleic acid contained in the complexes of the invention is mainly defined by the biological effect to be achieved in the cell, e.g. in the case of the use within the scope of gene therapy by the gene or gene section that is to be expressed or by the intended substitution or repair of a defect gene or any target sequence (Yoon et al., 1996; Kren et al., 1998), or by the target sequence of a gene to be inhibited (e.g. in the case of the use of antisense oligoribonucleotides or ribozymes). Preferably, the nucleic acid to be transported into the cell is plasmid DNA which contains a sequence encoding a therapeutically effective protein. For the use within the scope of cancer therapy, the sequence encodes, for instance, one or more cytokines such as interleukin-2, IFN-α, IFN-γ, TNF-α or for a suicide gene which is used in combination with the substrate. For the use in the so-called genetic tumour vaccination, the complexes contain DNA encoding one or more tumour antigens of fragments thereof, optionally in combination with DNA encoding one or more cytokines. Further examples of therapeutically effective nucleic acids are indicated in WO 93/07283.

The copolymer of the invention has the characteristic of sterically stabilising the nucleic acid-polycation complex and of reducing or inhibiting its undesired interaction with components of body fluids (e.g. serum proteins).

Suitable organic polycations for complexing nucleic acid for the transport into eukaryotic cells are known; due to their interaction with the negatively charged nucleic acid, it is compacted and put in a form suitable for being taken up by the cells. Examples thereof are polycations which were used for the receptor-mediated gene transfer (EP 0388 758; WO 93/07283) such as homologous linear cationic polyamino acids (such as polylysine, polyarginine, polyornithine) or heterologous linear mixed cationic-neutral polyamino acids (consisting of two or more cationic and neutral amino groups), branched and linear cationic peptides (Plank et al., 1999; Wadhwa et al., 1997), non-peptidic polycations (such as linear or branched polyethyleneimines, polypropyleneimines), dendrimers (speroidal polycations which can be synthesised with a well-defined diameter and an exact number of terminal amino groups; (Haensler and Szoka, 1993; Tang et al., 1996; WO 95/02397), cationic carbohydrates, e.g. chitosan (Erbacher et al., 1998). The polycations may also be modified with lipids (Zhou et al., 1994; WO 97/25070). Further suitable cations are cationic lipids (Lee et al., 1997) which are, in part, commerically available (e.g. Lipofectamin, Transfectam).

In the following, the term “polycation” is used as a substitute for both polycations and for cationic lipids, unless stated otherwise.

Within the meaning of the present invention, preferred polycations are polyethyleneimines, polylysine and dendrimers, e.g. polyamidoamine dendrimers (“PAMAM” dendrimers).

The size and/or charge of the polycations can vary to a large extent; it is chosen in a way that the complex formed with nucleic acid does not dissociate at a physiological salt concentration, which can easily be determined by means of the ethidium bromide displacement assay (Plank et al., 1999). In a further step, a defined amount of nucleic acid is incubated with increasing amounts of the polycation chosen, the complex formed is applied to the cells to be transfected and the gene expression (in general by means of a reporter gene construct, e.g. luciferase) is measured according to standard methods.

The formation of the nucleic acid complexes takes place via electrostatic interactions. In relation to the polycation, the DNA can be present in an excessive amount so that such complexes exhibit a negative surface charge; in the reverse case, i.e. if the polycation condensing the nucleic acid is present in an excessive amount, the complexes have a positive surface charge. Within the meaning of the present invention, the polycation is present in an excessive amount.

In the case of a positive charge surplus, the ratio of polycation and nucleic acid is adjusted so that the zeta potential is approximately +20 to +50 mV, if specific polycations, e.g. polylysine, are used, it may also be above said level. In the case of a negative charge surplus, the zeta potential amounts to approximately −50 to −20 mV.

The measurement of the zeta potential takes place according to established standard methods, such as described by e.g. Erbacher et al., 1998.

The polycation is optionally conjugated with a cellular ligand or antibody; suitable ligands are described in WO 93/07283. For the gene transfer directed to target cells during a tumour therapy, ligands or antibodies to tumour cell-associated receptors (e.g. CD87; uPA-R) are preferred which are able to increase the gene transfer into tumour cells.

During the production of the complexes, the nucleic acid - in general plasmid DNA—is incubated with the polycation (optionally derivatised with a receptor ligand) present in the charge surplus. During this process, particles are formed which can be taken up by the cells via receptor-mediated endocytosis. Subsequently, the complexes are incubated with a negatively charged copolymer according to the invention, preferably a polyethylene glycol copolymer. The effector E in the copolymer is preferred to be a polyanionic peptide. Alternatively, the copolymer is mixed with nucleic acid first and then incubated with polycation or, as a third variant, the copolymer is mixed with polycation first and then incubated with nucleic acid.

Alternatively, the nucleic acid is incubated with a polycation present in the electrostatic deficit and then a cationic copolymer is added. In this case, too, the order of the mixing steps can be varied as described for anionic copolymers, above. The relative portions of the individual components are chosen in a way that the resulting DNA complex exhibits a weak positive, neutral or weak negative zeta potential (+10 mV to −10 mV).

If positively charged copolymers are used, they can be used as the only polycationic molecules binding and condensing nucleic acid; thus, the portion of a polycation or cationic lipid is not necessary. In this case, too, the relative portions of the individual components are chosen in a way that the resulting DNA complex exhibits a weak positive, neutral or weak negative zeta potential (+10 mV to −10 mV).

In the complexes, optionally, polycations and/or copolymers are modified with identical or different cellular ligands.

The nucleic acid complexes according to the invention, which are stabilised in their size by the electrostatically-bound copolymer of the general formula I and, thus, protected against aggregation, have the advantage that they can be stored in solution over long periods of time (weeks). Furthermore, they have the advantage that they do not interact or interact to a lower extent with components of body fluids (e.g. with serum proteins) due to the protective effect of the copolymer bound.

In a further aspect, the invention relates to a pharmaceutical composition containing a therapeutically effective nucleic acid, the copolymer according to the invention and, optionally, an organic polycation or cationic lipid.

The pharmaceutical composition according to the invention is preferred to be present in lyophilised form, optionally supplemented by sugar such as sucrose or dextrose in an amount which results in a physiological concentration in the solution ready for use. The composition can also be present in the form of a cryoconcentrate. The composition according to the invention can also be present in a deep-frozen (cryopreserved) form or as a cooled solution.

In a further aspect, the positively-charged or negatively-charged copolymers according to the invention serve the purpose to sterically stabilise colloidal particles (“nanoparticles”) as developed for the application of classic pharmaceutical preparations and to reduce or suppress their undesired interaction with components of body fluids (e.g. with serum proteins). Furthermore, the copolymers according to the invention modified with receptor ligands can be used for attaching receptor ligands to the surface of said nanoparticles to transfer drugs with increased specificity to target cells (“drug targeting”).

In a further aspect, the present invention relates to a combination of a carrier and a complex containing one or more nucleic acid molecules and one or more copolymers according to the invention.

With regard to the preferred embodiments of the copolymers and the nucleic acid molecules, the explanations above apply.

In this context, a carrier is a body or a substance which can be contacted in vivo or in vitro with cells to be transformed and which carries the complex of nucleic acid(s) and copolymer(s). Preferably, the carrier is a material connected in a coherent way, i.e. a solid substance, particularly preferably a plastic or deformable solid substance such as e.g. a gel, a sponge, a foil, a powder, a granulate or a fascia. The carrier can consist of biologically non-resorbable or biologically resorbable material.

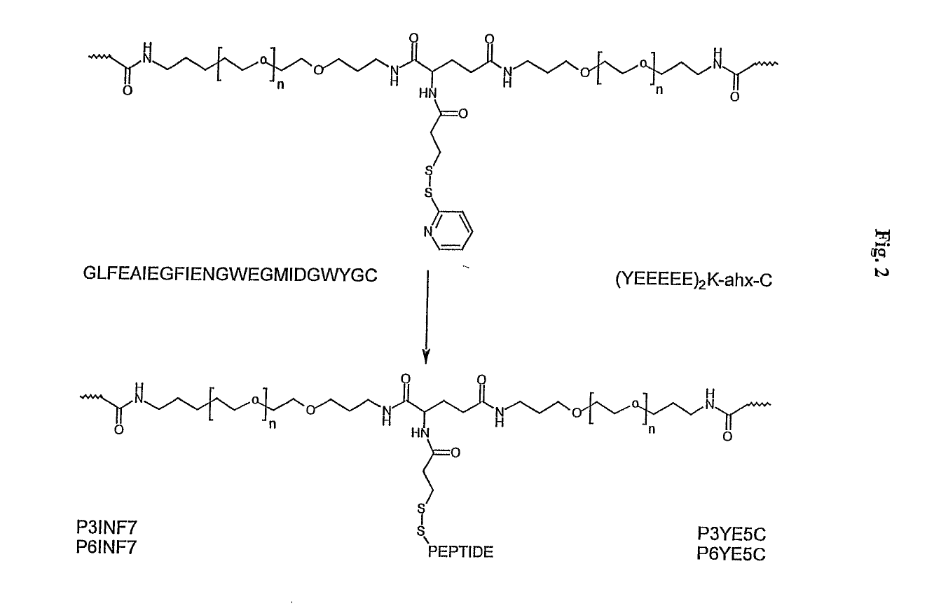

The carrier may also be a carrier produced by the cross-linkage of the copolymers according to the invention, preferably in the presence of nucleic acid molecules. Thus, there is, for example, the possibility of introduction of known gene vectors (naked DNA, naked RNA, lipoplexes, polyplexes) and of oligonucleotides and ribozymes, optionally chemically modified, in cross-linked polymers according to the invention. For this purpose, the cross-linkage takes place, e.g. in situ in the presence of the gene vector, DNA, oligonucleotide etc. by addition of an agent triggering the cross-linkage in an aqueous or organic solvent. The nature of the cross-linking agent depends on the structure of the copolymer. Therefore, e.g. the polymer backbone shown in FIG. 2 can be cross-linked by addition of dithiols such as e.g. cyteinyl-cysteine¹ or non-aminoacid-like dithiols. Cross-linkage of copolymers containing carboxylic acid can take place by adding any diamines during the activation of carboxylic acid (e.g. reaction of the carboxylic acid to an activated ester in situ) (Nathan et al., Macromolecules 25 (1992), 4476-4484). A polymer backbone with primary or secondary amines can take place e.g. by adding an activated dicarboxylic acid. After the cross-linkage, the preparation can be dried until a film is formed.

An example of a biologically non-resorbable material is silicon (e.g for catheters). It is, however, also possible to use different biologically non-resorbable materials which can be introduced into the body as implants and/or have already been used, e.g. in ¹translator's note: “cyteinyl-” reflects a typing error in the German original and should actually read “cysteinyl-”. plastic surgery. Examples thereof are PTFE (e.g. for vessel replacements), polyurethane (e.g. for catheters), metal materials (e.g. medicinal steels, titat alloy² for endoprostheses; metal meshes to be used as vessel support (stents)).

Preferably, the carrier is a biologically resorbable material. Examples thereof are fibrin glues produced from thrombin or fibrinogen, chitin, oxycellulose, gelatine, polyethylene glycol carbonates, aliphatic polyesters such as e.g. polylactic acids, polyglycol acids and the amino acid compounds derived therefrom, such as polyamides and polyurethanes or polyethers and the corresponding mixed polymerisates. Moreover, any other biologically degradable polymer can be used as carrier, in particular so-called self-curing adhesives on the basis of hydrogels. In particular, any materials are suitable as biologically resorbable materials which can be degraded enzymatically in the body and/or by hydrolytic processes. Examples thereof are also bio-resorbable chemically defined calcium sulphate, tricalcium phosphate, hydroxy apatite, polyanhydride, carriers made out of purified proteins or of partially purified extracellular matrix. The carrier collagen is particularly preferred, particularly preferably a collagen matrix produced from cartilage and skin collagens, as distributed e.g. by Sigma or Collagen Corporation. Examples of the production of a collagen matrix are described e.g. in the U.S. Pat. Nos. 4,394,370 and 4,975,527. The carrier is very much preferred to be from collagen and particularly preferred to be a collagen sponge. In general, negatively charged polysaccharides such as glucosaminoglycans bind to collagen via ionic interactions. The binding can take place to positively charged amino acids in the collagen fibrils (lysine, hydroxylysine and arginine) or even to negatively charged amino acids, mediated by divalent cations such as calcium. Furthermore, the ionic binding properties of collagen can purposefully be influenced by pre-treatment with acid or alkaline solution and subsequent freeze-drying. By means of these techniques known in collagen chemistry it is possible to soak collagen materials with suspensions of complexes according to the invention to produce an ionic binding between collagen as carrier material and the DNA complexes. 2 translator's note: “titat” reflects a tying error in the German original and should actually read: “titan”.

In collagen, positively charged amino acids are not concentrated in short cationic sections. Such structural features of the carrier, however, are necessary for the efficient binding of DNA. In order to achieve a tighter binding to the carrier material, the latter can further be derivatised with cationic substances binding DNA such as peptides (Plank et al., Human Gene Therapy 10 (1999), 319-333) or polyethyleneimine (PEI). For this purpose, the collagen sponge is modified e.g. with the bifunctional coupling reagent succinimidyl-pyridyl-dithiopropionate (SPDP). Polyethyleneimine is derivatised with iminothiolane which leads to the introduction of thiol groups. The cationic peptide to be coupled carries a cysteine at the C-terminus. The thiol groups react with the SPDP-derivatised collagen sponge by forming disulphide bridges. The sponge derivatives obtained in that manner should bind the DNA tightly, and the release of the DNA is to be expected to take place with a long delay in time.

For the production of a combination according to the invention, for example, the dry collagen material can be incubated with DNA/copolymer complexes in 5% glucose. The sponges are then freeze-dried.

In general, a combination according to the invention can be produced by contacting a corresponding carrier with the complex of nucleic acid and copolymer so that the carrier absorbs the complex or binds it in such a way that it can be released again. Corresponding methods are known to the person skilled in the art (Bonadio et al. (1999). Nat. Med. 5(7): 753-759; Shea, L. D. et al. (1999). Nat. Biotechnol. 17 (6): 551-554). In the Examples, the production of a combination of collagen sponge as carrier and a nucleic acid/copolymer complex is described.

The combinations according to the invention can be used for the transfer of nucleic acids into cells, preferably into cells of higher eukaryotes, preferably of vertebrates, particularly of mammals both in vitro and in vivo.

In connection with the in vivo application, it is possible, in particular, to introduce the combination directly as an implant, e.g. subcutaneously or as coating e.g. on a catheter, joint replacement or an endoprosthesis (e.g. for the improvement of tissue integration). Further possible applications are wound coverages, general the coverage of extensive-skin defects such as e.g. with burns or decubital ulcers, and as carrier material for the modern techniques of tissue engineering (Mooney, D. J. and Mikos, A. G. (1999). Sci. Am. 280(4): 60-65). Furthermore, processing of the coated materials is possible in form of powders which are purposefully introduced into and fixed in the organism by means of common tissue glue systems and become effective in the form of a depot (transfection).

Moreover, the present invention also relates to a pharmaceutical composition containing a combination according to the invention, optionally in connection with pharmaceutically acceptable additives.

A kit containing a carrier as defined above as well as a copolymer according to the invention or a complex of a copolymer according to the invention and a nucleic acid molecule is also subject matter of the invention.

FIG. 1: Preparation of the copolymer backbones from 3-(2′-thiopyridyl)-mercaptopropionyl-glutamic acid and O,O′-bis(2-aminoethyl)poly(ethylene glycol) 6000 or O,O′-bis(2-aminoethyl)poly(ethylene glycol) 3400

FIG. 2: Coupling of charged peptides to the copolymer backbone

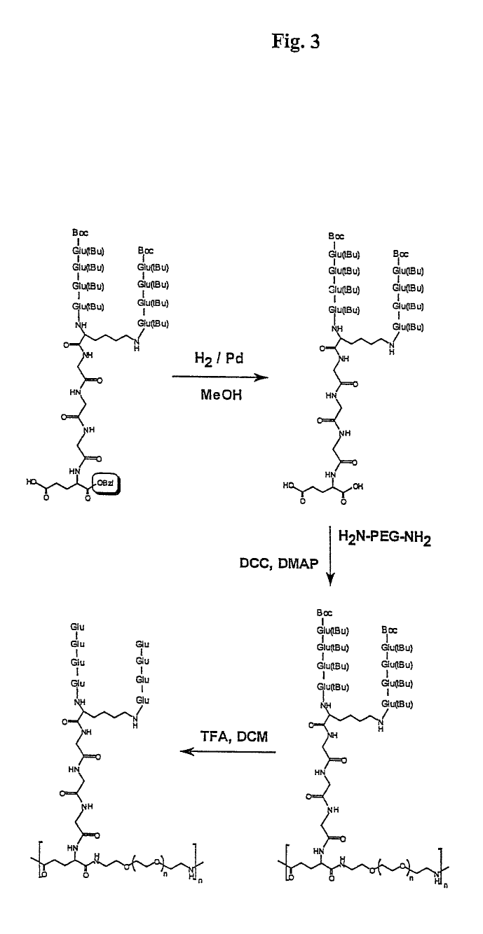

FIG. 3: Preparation of the copolymer backbone from the protected peptide E4E^{PROT} and O,O′-bis(2-aminoethyl)poly(ethylene glycol) 6000

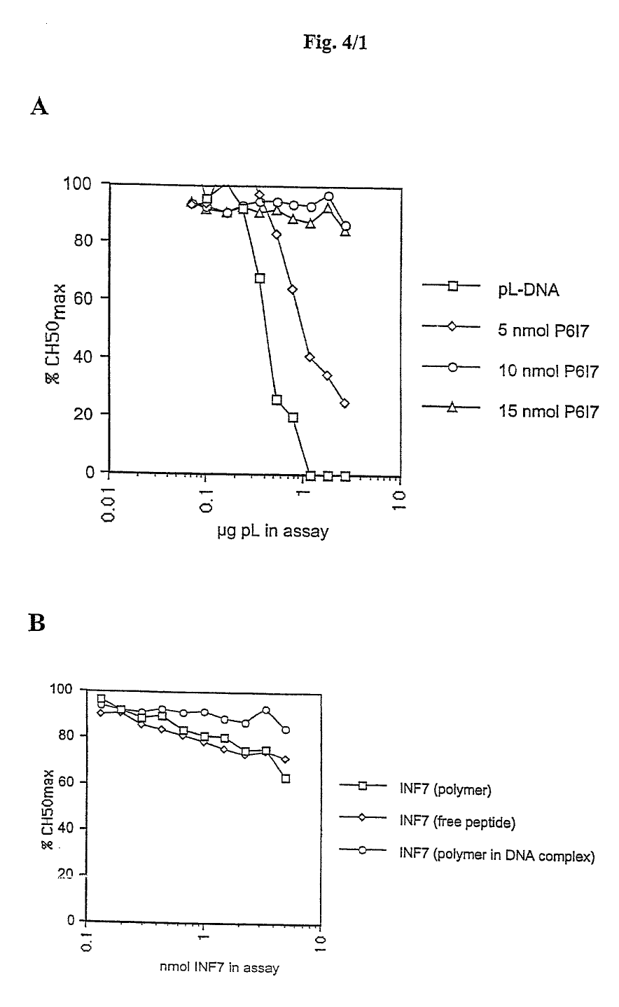

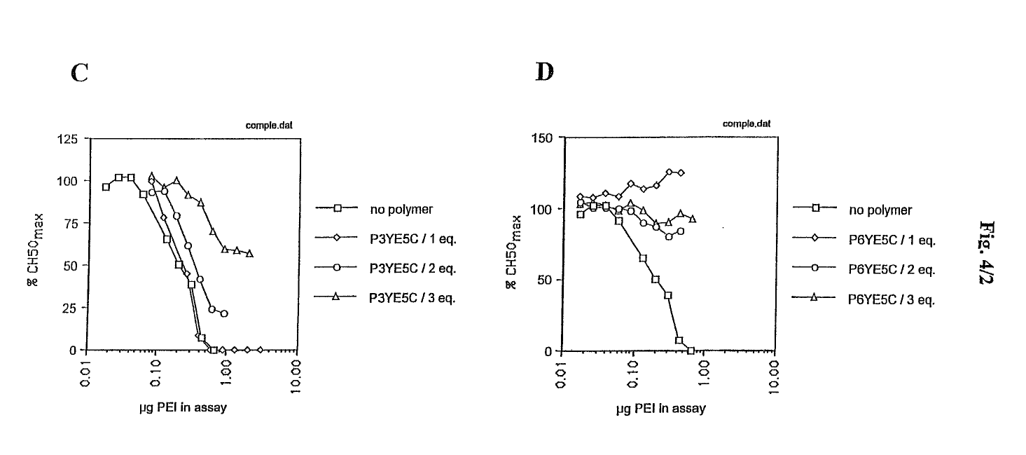

FIG. 4: Complement activation assays

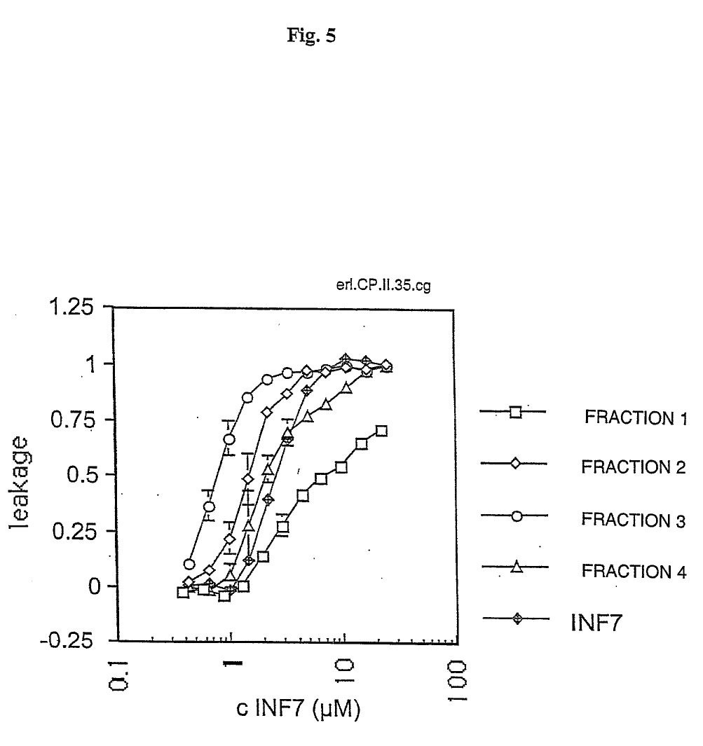

FIG. 5: Erythrocyte lysis assay

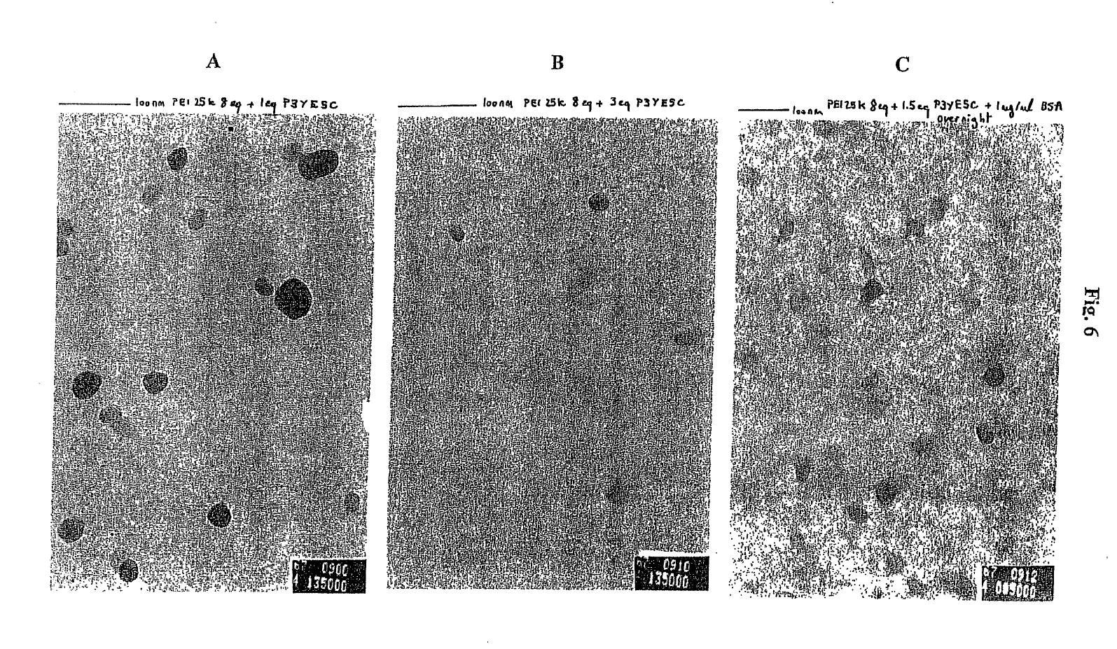

FIG. 6: Electron micrographs of PEI-DNA complexes (N/P=8) in the presence of the copolymer P3YE5C

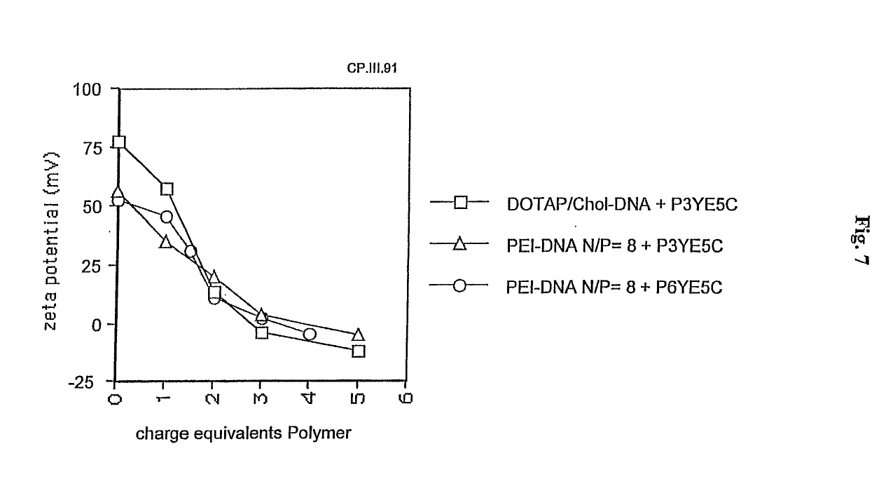

FIG. 7: Zeta potential of PEI- and DOTAP/cholesterol-DNA complexes in dependence of the amount of added copolymers P3YE5C and P6YE5C, respectively

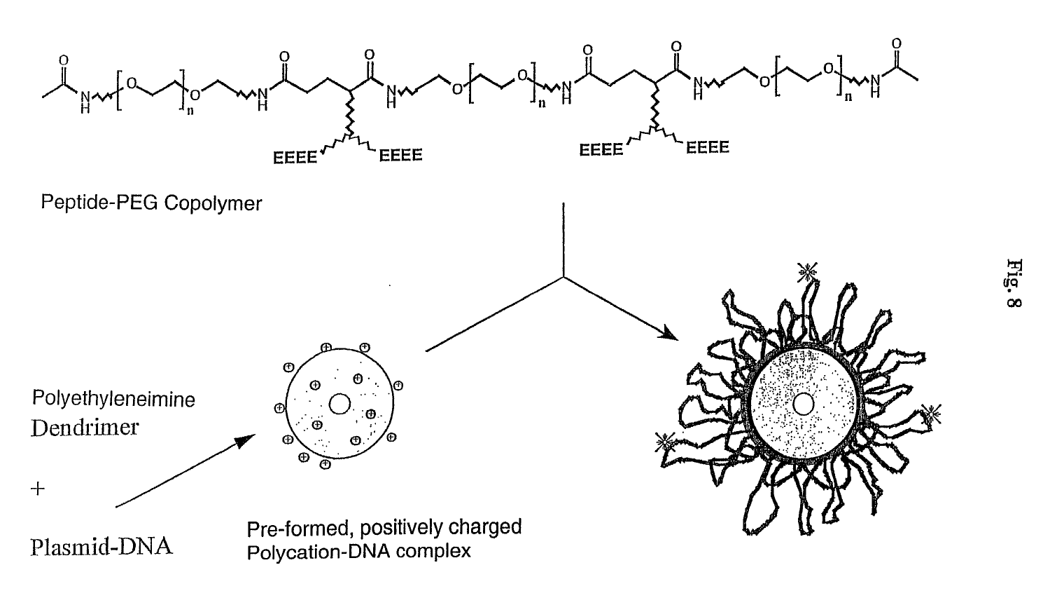

FIG. 8: Preparation of DNA/polycation/copolymer complexes

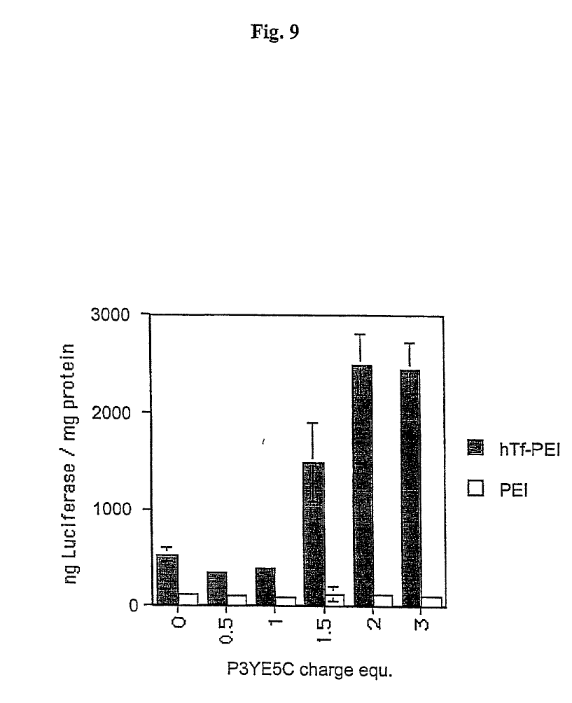

FIG. 9: Gene transfer into K562 cells with PEI(25 kD)-DNA complexes in the presence and in the absence of the copolymer P3YE5C

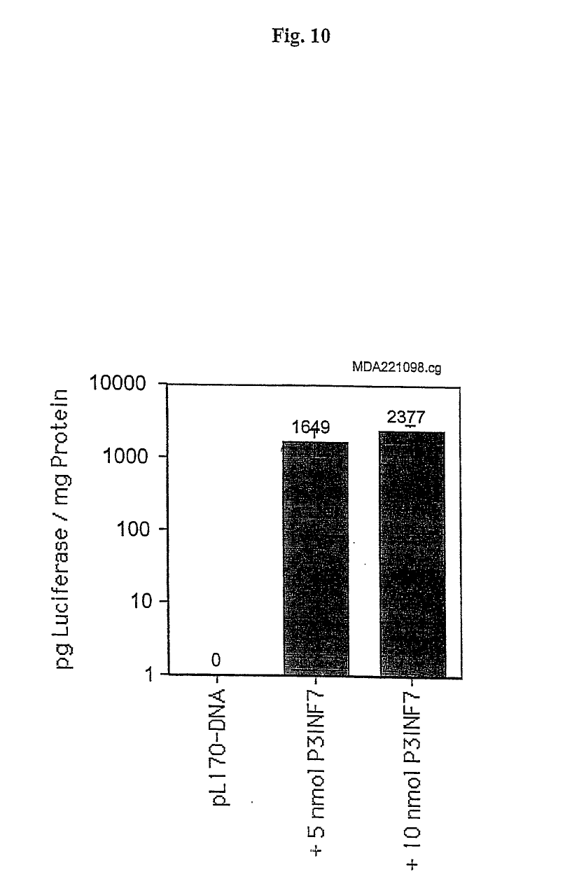

FIG. 10: Transfection of the mamma carcinoma cell line MDA-MB435S with polylysine-DNA complexes in the presence and in the absence of the coating polymer P31NF7

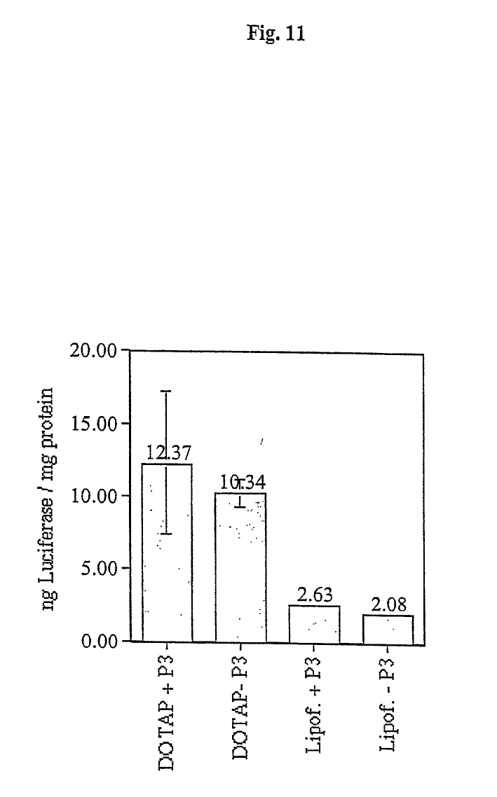

FIG. 11: Lipofection in NIH3T3 cells in the presence and in the absence of the copolymer P3YE5C

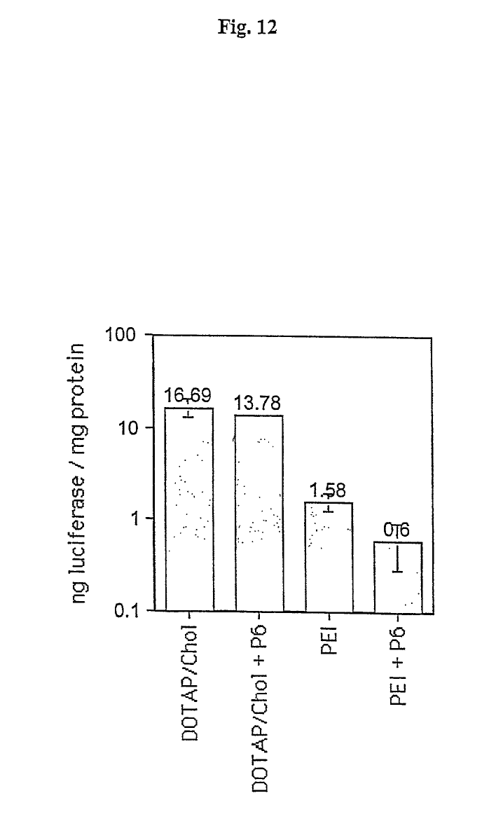

FIG. 12: Transfection of HepG2-cells with DOTAP/cholesterol-DNA and PEI-DNA in the presence and in the absence of P6YE5C

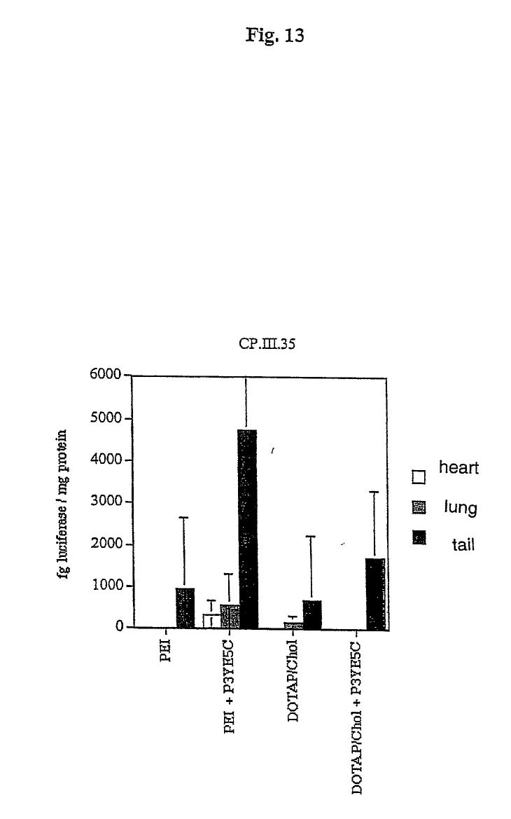

FIG. 13: Intravenous gene transfer in vivo with DNA/polycation complexes with a copolymer coating

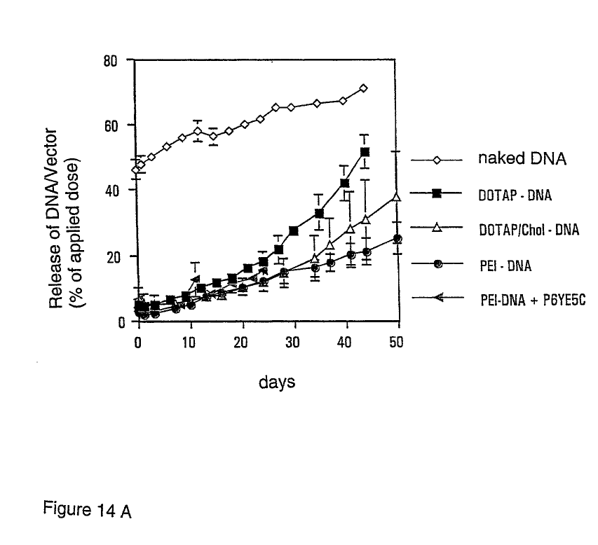

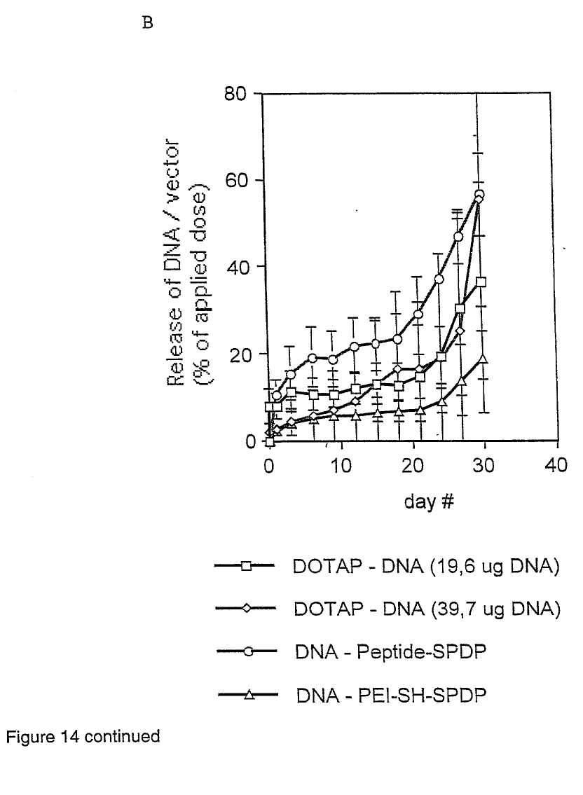

FIG. 14: Release of radioactive-labeled DNA from vector-loaded collagen sponges. The sponges were prepared as described in Example 17. In the case of naked DNA, approximately 50% of the applied dose bind actively, whereas the other half is immediately released. The subsequent release kinetics follows an approximately linear course. If gene vectors are loaded on sponges, a fraction of 90% is bound tightly and is released over an extended time period with an exponential release profile. Cationically derivatized sponges (“PEI-SPDP” and “Peptide-SPDP”) bind naked DNA efficiently and display release kinetics similar to vector-loaded sponges.

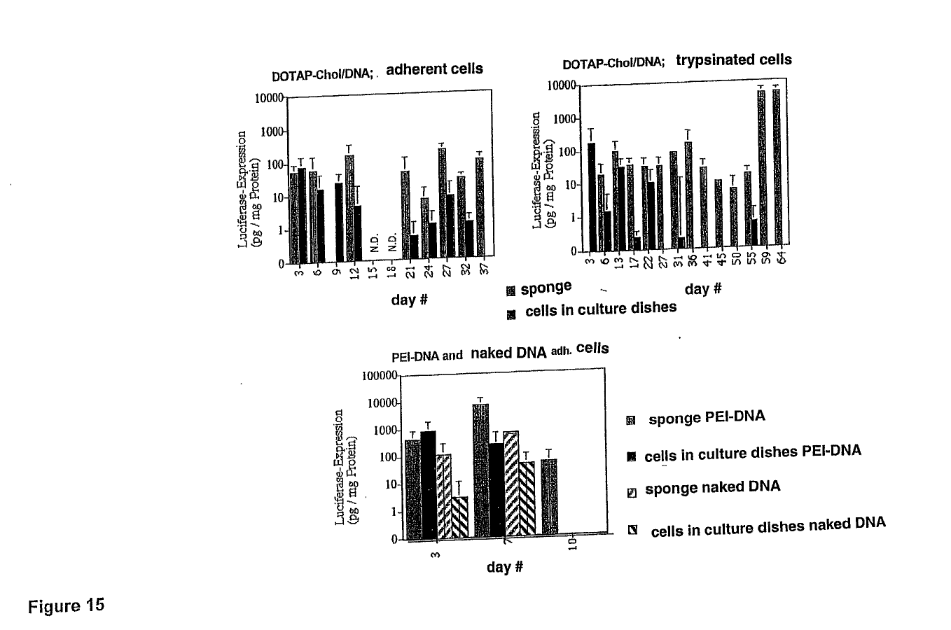

FIG. 15: Gene transfer into NIH3T3 mouse fibroblasts by vector-loaded collagen sponges. The sponges were prepared as described in Example 16 (naked DNA, PEI-DNA, DOTAP-cholesterol-DNA prepared according to the variant procedure) and used for gene delivery as described in Example 18. In the case of DOTAP-cholesterol sponges, the preparations were either added to an adherent layer of cells (left), or freshly trypsinized cells were loaded on the sponge (right). The subsequent experimental course was identical for all setups. The reporter gene expression was assayed over various time spans and persists over extended periods particularly in cells growing on/in the sponges.

### EXAMPLE 1

### Preparation of Charged Copolymers of the General Formula I

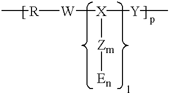

1.1. Preparation of the Copolymer Backbones From 3-(2′-Thiopyridyl)-Mercaptopropionyl-Glutamic Acid and O,O′-Bis(2-Aminoethyl)Poly(Ethylene Glycol) 6000 or O,O′-Bis(2-Aminoethyl)Poly(Ethylene Glycol) 3400 (Diamino-PEG-3400; Fluka)

In this case, in the general formula I is:

W═Y═NH;

X=3-mercaptopropionyl-glutamic acid, that is, an amino acid derivative according to case i) which was derived by coupling of the linker moiety 3-(2′-thiopyridyl)-mercaptopropionic acid to glutamic acid; hence, Z is omitted (m=0).

a) Reaction of 3-mercaptopropionic acid with 2,2′-dithiodipyridine (1):

1 g DTDP (Fluka) was dissolved in 4 ml absolute ethanol (Merck). After addition of 100 μl triethyl amine (Aldrich), 87 μl (1 mmol) 3-mercaptopropionic acid were added. After 1 h, the reaction mixture was separated in aliquots by reverse phase HPLC: preparative C18-column (Vydac, 218TP1022), flow rate 25 ml/min, 0.1% trifluoroacetic acid, 0-40% acetonitrile in 24 min, 40-100% acetonitrile in 5 min, 100% acetonitrile during 5 min. The product peak eluted with ca. 20% acetonitrile. The product fractions were pooled and lyophilized. In a variant of this protocol, excess DTDP is precipitated prior to RP-HPLC purification by slow addition of water while stirring. The precipitate is redissolved twice in ethanol and re-precipitated by addition of water. The combined aqueous phases are purified by RP-HPLC as described above.

b) Synthesis of 3-(2′-thiopyridyl)-mercaptopropionyl-glutamic acid (2b):

Product 1, obtained in a) (see FIG. 1; 0.5 mmol) was dissolved in 25 ml dichloromethane. One mmol each of glutamic acid-di-t-butyl ester (Glu(OtBu)OtBu, Bachem), 1-hydroxybenzotriazole (Aldrich), N-ethyl-N′-(dimethylaminopropyl)-carbodiimide (Aldrich) and diisopropylethylamine (Aldrich) were added in a 50 ml polypropylene tube in a stepwise manner while stirring and cooling on ice. After 48 h reaction, the mixture was reduced to an oily residue by rotary evaporation. The residue was taken up in 20 ml ethyl acetate. This solution was extracted twice each with 0.5 M hydrochloric acid, saturated sodium hydrogencarbonate solution and saturated sodium chloride solution. The organic phase was reduced to an oily residue by rotary evaporation and dried over night under high vacuum (product 2a; see FIG. 1). For the removal of the t-butyl protecting groups, product 2a was redissolved without further purification in 30 ml dichloromethane: trifluoroacetic acid (2:1) and stirred for 2 h at room temperature. The solution was reduced to an oily residue on a rotary evaporator, which was subsequently washed with ice-cold ether. After drying under high vacuum, the product was dissolved in 100 mM HEPES pH 7.4 and purified in aliquots by RP-HPLC (same conditions as for product 1). The product fractions were pooled. Product 2b (see FIG. 1) was obtained with a yield of 270 μmol (27% over all steps). Calculated molecular weight: 344.05. Found: 345,0 (MH⁺).

c1) Copolymerisation of pyridyl-(2-dithiopropionyl)-glutamic acid (2b) with O,O′-bis(2-aminoethyl)poly(ethylene glycol) 6000 (diamino-PEG-6000; Fluka)

Produkt 2b was dissolved in 3 ml dimethylformamide (Fluka) and diluted to 20 ml with dichloromethane. 5 ml of this solution (67.5 μmol) were mixed in a stepwise manner with 506 mg diamino-PEG-6000 (84 μmol, corresponding to 1.25 equivalents; Fluka), 30 mg dicyclohexylcarbodiimide (135 μmol, 2 equivalents, 135 μl of a 1 M solution in DMF) and 2 mg dimethylaminopyridine (0.25 equivalents, 1 M solution in DMF). After 2 h, 10 μl were removed for a ninhydrin assay, which produced only a faint blue staining. Raw product 3 (see FIG. 1) was obtained by precipitation from the reaction mixture with t-butyl-methylether after cooling to −20° C. while stirring. The product was dried in vacuo. Aliquots were dissolved in water and purified by gel filtration after removal of a non-soluble residue by filtration (Ultra-Free MC, Millipore). For this purpose, an XK 16/40-column (Pharmacia) was filled with Superdex 75 (Pharmacia) according to the recommendations of the manufacturer. Aliquots of 20 mg each of raw product 3 were purified at a flow rate of 1 ml/min with 20 mM HEPES pH 7.3 as eluent. The main fraction eluted with an apparent molecular weight of 40.000 Da after preceding, clearly separated fractions of higher molecular weights which were collected separately.

c2) Copolymerization of pyridyl-(2-dithiopropionyl)-glutamic acid (2b) with O,O′-bis(2-aminoethyl)poly(ethylene glycol) 3400 (diamino-PEG-3400; Fluka) (product 4; see FIG. 1) Product 4 was obtained with the same setup and purification procedures as product 3. A product was isolated as the main fraction (54% of all fractions) after gel filtration, eluting with an apparent molecular weight of 22.800 Da (side fractions were a product of 64 kD, 14% of the total, and a product of 46 kD, 32% of the total). The reaction scheme for the synthesis steps a) to c), yielding the copolymer backbone, is shown in FIG. 1: 3-mercaptopropionic acid is reacted with 2,2′-dithiodipyridine. Product (1) is coupled to carboxyl-protected glutamic acid (product 2a). After cleavage of the t-butyl protecting groups, 3-(2′-thiopyridyl)-mercaptopropionyl-glutamic acid (2b) is obtained, which is copolymerized under DCC activation with O,O′-bis(2-aminoethyl)poly(ethylene glycol) 6000 or with O,O′-bis(2-aminoethyl)poly(ethylene glycol) 3400. The procedure yields products 3 and 4, respectively.

1.2. Peptide Synthesis

The peptides were synthesized according to the FaStMOc™ protocol using an Applied Biosystems 431A peptide synthesizer.

i) Peptide YE5C (sequence [Ac-YEEEEE]₂-ahx-C) was synthesized using 330 mg cysteine-loaded chlorotrityl resin (0.5 mmol/g; Bachem) using the protecting groups trityl- (Cys), di-Fmoc (Lys) and O-t-butyl- (Glu). 1 mmol each of protected amino acids were used. After the branching point (Lys), double couplings were carried out. The acetylation of the N-termini was carried out on the resin-coupled peptide using 2 mmol acetic anhydride in 2 ml N-methylpyrrolidone in the presence of 2 mmol diisopropylethylamine. The peptide was obtained as raw product after cleavage from the resin (500 μl water, 500 μl thioanisole, 250 μl ethanedithiol in 10 ml trifluoroacetic acid) and precipitation with diethylether. The raw product was dissolved in 100 mM HEPES pH 7.9 and purified by perfusion chromatography (Poros 20 HQ, Boehringer Mannheim, filled into a 4×100 mm PEEK column. 0-0.5 M NaCl in 8 min, flow rate 10 ml/min). The extinction coefficient of the peptide in 50 mM sodium phosphate buffer in 6 M guanidinium hydrochloride at 280 nm is 2560 M⁻¹cm⁻¹ (Gill and von Hippel 1989).

ii) Peptide INF7 (sequence GLFEAIEGFIENGWEGMIDGWYGC) was synthesized according to the same procedure on 500 mg chlorotrityl resin (0.5 mmol/g), cleaved from the resin as described for YE5C and precipitated with diethyl ether. The raw product was dried in vacuo. Aliquots of 20 mg each were dissolved in 500 μl 1 M triethylammonium hydrogencarbonate buffer pH 8 and purified by gel filtration (Sephadex G-10 from Pharmacia filled into a HR 10/30 column from Pharmacia. Flow rate 1 ml/min. Eluent: 20 mM HEPES pH 7.3/150 mM NaCl or 100 mM TEAB or 100 mM ammonium hydrogencarbonate). Extinction coefficients: 278 nm 12600; 279 nm 12665; 280 nm 12660 M⁻¹cm⁻¹.

iii) Peptide SFO29-ahx (Sequence K₂K-ahx-C) was synthesized in analogous manner (500 mg Fmoc-Cys(Trt)-Chlorotrityl resin, Bachem; 0.5 mmol/g) and purified according to standard procedures (Sephadex G10 with 0.1% TFA as eluent; reverse phase HPLC, 0.1% TFA—acetonitrile gradient). The lysine at the branching point was alpha,epsilon-di-Fmoc-L-lysine, the subsequent lysines were alpha-Fmoc-epsilon-Boc-L-lysine.

iv) Peptide E4E (sequence [EEEE]₂KGGE) was synthesized in analogous manner. Synthetic scale: 0.25 mmol Fmoc-Glu(OBzl)-Chlorotrityl resin. The loading of the resin was carried out by suspension of the corresponding amounts of O-chlorotritylchloride resin (Alexis) in absolute dichloromethane and mixing with 2 eq. each of Fmoc-Glu(OBzl)OH and diisopropylethylamine. After shaking for several hours, the resin was filtrated and washed several times with dimethylformamide, methanol, isopropanol, dichloromethane and diethylether. A modified Fmoc-protocol is used. The N-terminal amino acid carries a Boc protecting group to yield a fully protected, base-stable peptide derivative from the solid phase synthesis with the sequence (E(Boc)[E(tBu)]₃)₂KGGE(OBzl)OH (E4E^{PROT}).

The cleavage from the resin was carried out with dichloromethane/acetic acid/trifluoroethanol 8:1:1 at room temperature. The benzylester protecting group of the C-terminal glutamic acid was selectively removed with H₂/palladium on activated charcoal according to standard procedures.

Peptide masses were determined by electrospray mass spectroscopy which confirmed the identity of the peptides.

1.3. Coupling of the Peptides to the Copolymer Backbones (4) and (5), Respectively

The solutions in 20 mM HEPES, pH 7.4 of 1.2 equivalents (with respect to the thiopyridyl groups in the polymer) of C-terminal cysteine-containing peptide and copolymer backbone, obtained in 1.1, are mixed and shaken or stirred for 15 h at room temperature.

For the determination of the equivalents to be used, the available thiopyridyl coupling sites are determined by reaction of a diluted polymer solution with 2-mercaptoethanol and subsequent measuring of the absorbance of released 2-thiopyridone at a wavelength of 342 nm. The concentration of the free thiol groups of the cysteine-containing peptide is determined with Ellman's reagent at a wavelength of 412 nm according to Lambert-Beer.

After completeness of the reaction, which was determined by the absorbance of released thiopyridone at 342 nm, the volume of the reaction mixture was reduced and the product was fractionated by gel filtration (Superdex 75, Pharmacia).

1.3.1 Preparation of the Copolymer P3YE5C

The branched peptide YE5C, sequence (YEEEEE)₂K(ahx)C, was used which is coupled via a disulfide bridge of the cysteine thiol to the 3-mercaptopropionyl-glutamic acid group.

a) The copolymer P3YE5C was prepared from fraction 3 (22.800 Da) of product (4) and purified peptide. As a product a compound was obtained with an apparent molecular weight of 35.000 Da. With respect to the molecular weight of the peptide and the copolymer backbone used, this means a degree of polymerization of p=6 (6 repeating units).

b) The copolymer P6YE5C was prepared from fraction 3 (40.200 Da) of product (3) and purified peptide. As a product a compound was obtained with an apparent molecular weight of 55.800 Da. The degree of polymerization is approximately 7.

1.3.2 Preparation of the Copolymer P3INF7

The endosomolytic peptide INF7 was used, which is coupled via a disulfide bridge of the cysteine thiol to the 3-mercaptopropionyl-glutamic acid group.

a) Copolymer P31NF7 was prepared from fraction 3 (22.800 Da) of product (4) and purified influenza peptide.

b) Copolymer P6INF7 was prepared from fraction 3 (40.200 Da) of product (3) and purified influenza peptide INF 7.

1.3.3 Preparation of a Receptor Ligand-modified (“Lactosylated”) Copolymer

One part of lactosylated peptide SFO29-ahx and 9 parts of the branched peptide YE5C were used, which were coupled via a disulfide bridge of the cysteine thiols to the 3-mercaptopropionylglutamic acid groups. 3.32 μmol each (with respect to the inherent thiopyridyl groups) of copolymer (4) and (5), respectively, dissolved in 1 ml 20 mM HEPES pH 7.4 were incubated with a mixture of 500 nmol lactosylated SFO29-ahx and 4.48 μmol peptide YE5C in 1.1 ml HEPES buffer. This corresponds to a 1.5-fold excess of free thiol groups from the peptides over the available thiopyridyl groups. The fraction of lactosylated peptide among total peptide is 10%. The reaction proceeded quantitatively over night. The products were purified by gel filtration (Superdex 75) as described.

The rection scheme of the peptide coupling to the copolymer backbone according to 1.3 is shown in FIG. 2. Peptides with free thiol groups are coupled to products (3) or (4), respectively, for example the peptide INF7 (left) or the peptide YE5C. This yields the products P3INF7 (prepared from O,O′-bis(2-aminoethyl)poly(ethylene glycol) 3400), P6INF7 (prepared from O,O′-bis(2-aminoethyl)poly(ethylene glycol) 6000) and in an analogous manner P3YE5C and P6YE5C.

### EXAMPLE 2

### Preparation of the Copolymer Backbone Fmoc-6-aminohexanoyl-glutamic Acid and O,O′-bis(2-aminoethyl)poly(ethylene Glycol) 6000 (Diamino-PEG-6000; Fluka) or O,O′-bis(2-aminoethyl)poly(ethylene glycol) 3400 (Diamino-PEG-3400; Fluka)

In this case, in the general formula I:

W═Y═NH; X═Fmoc-6-aminohexanoyl-glutamic acid.

This means, X according to i) is an amino acid derivative which is obtained by coupling of Fmoc-6-aminohexanoic acid to glutamic acid. For the coupling of the effector molecule E, Z can be omitted or can be a bifunctional linker such as SPDP or EMCS.

An effector suitable for coupling to the polymer backbone can be a peptide of the type E4E^{PROT} (Z is omitted) or of the type YE5C. In the latter case, the peptide reacts via its cysteine thiol with a linker molecule Z (such as SPDP or EMCS).

a) Synthesis of the di-peptide Fmoc-6-aminohexanoic acid-GluOH (6):

1 g of Fmoc-protected 6-aminohexanoic acid (2.82 mmol), 1.2 eq. Glu(OtBu)OtBu and 1.2 eq. 1-hydroxybenzotriazole are dissolved in 200 ml dichloromethane. Upon cooling to 0° C., 1.2 eq. N-ethyl-N′-(dimethylaminopropyl)-carbodiimide and 1.7 ml diisopropylethylamine were added to the mixture (pH=8). After one hour at 0° C., the mixture was stirred for 18 hr at room temperature. The solvent was completely removed by distillation, the residue was taken up in ethyl acetate and extracted with 0.5 N hydrochloric acid, saturated sodium hydrogencarbonate solution and saturated sodium chloride solution. After evaporation of the solvent, Fmoc-6-aminohexanoyl-Glu(OtBu)OtBu (5) was yielded upon lyophilization. Di-t-butyl-protected derivative (5) was dissolved in 30 ml dichloromethane/trifluoroacetic acid 2:1 and stirred for one hour at room temperature. Upon completeness of reaction (assessed by reverse phase-HPLC), the solvent was reduced to approximately 5% of the initial volume. Product (6) was yielded upon precipitation from diethyl ether. The final purification was carried out by RP-HPLC with an acetonitrile/water/ 0.1% TFA gradient.

b) Copolymerization of Fmoc-6-aminohexanoic acid-GluOH (6) with O,O′-bis(2-aminoethyl)poly(ethylene glycol) 3400′ (diamino-PEG-3400, Fluka), product (7):

10 mg (6), 1.5 eq. O,O′-bis(2-aminoethyl)poly(ethylene glycol) 3400′, 2 eq. dicyclohexylcarbodiimide and 0.25 eq. 4-(dimethylamino)-pyridine are dissolved in 5 ml dichloromethane. After stirring for 30 min at room temperature and reducing its volume, the solution was filtered followed by complete removal of the solvent by distillation. The residue was suspended in 500 μl of water and lyophilized.

After removal of the Fmoc protecting group (20% piperidine in dimethylformamide or dichloromethane) from the polymer, the copolymer can be conjugated by standard peptide coupling chemistry with any peptide displaying a free C-terminus .

### EXAMPLE 3

### Preparation of the Copolymer Backbone from the Protected Peptide E₄E^{PROT} and O,O′-bis(2-aminoethyl)poly(ethylene glycol) 6000 (diamino-PEG-6000; Fluka) or O,O′-bis(2-aminoethyl)poly(ethylene glycol) 3400 (diamino-PEG-3400; Fluka)

In this case, in the general formula I:

W═Y═NH; X=the branched peptide E4E^{PROT}.

In this example, a polyanionic pepitde X according to i) itself represents the effector. Therefore, Z and E are omitted (m=n=0)

Copolymerization of E4E^{PROT} with O,O′-bis(2-aminoethyl)-poly-(ethylene glycol) 6000′ (diamino-PEG-6000, Fluka) (8);

50 μmol E4E^{PROT}, 1.5 eq. O,O′-bis(2-aminoethyl)-poly(ethylene glycol) 6000′, 2 eq. dicyclohexylcarbodiimide and 0.25 eq. 4-(dimethylamino-)pyridine were dissolved in 10 ml dichloromethane. After stirring at 4° C. for four hours and after reducing its volume, the solution was filtered followed by complete, removal of the solvent by distillation. The residue was suspended in 500 μl water and lyophilized.

For the cleavage of the remaining acid-labile side chain protecting groups, trifluoroacetic acid containing up to 5% scavenger (preferably ethane dithiol, triethylsilane, thioanisol) was added according to procedures described in the literature followed by stirring for 2 hours. The raw product was isolated by precipitation from diethyl ether. The final purification was carried out by gel filtration (Superdex 75, Pharmacia) as described above.

FIG. 3 shows the reaction scheme: The benzyl protecting group on carboxylate 1 of the C-terminal glutamic acid of the fully protected peptide E4E^{PROT} is selectively cleaved by H₂/Palladium on activated charcoal. The product is co-polymerized upon DCC activation with O,O′-bis(2-aminoethyl)poly(ethylene glycol) 6000 or with O,O′-bis(2-aminoethyl)poly(ethylene glycol) 3400. In the final step, the protecting groups of the N-terminally positioned glutamic acids are cleaved with TFA in DCM.

### EXAMPLE 4

### Complement Activation Studies

The assay was carried out essentially as described in Plank et al., 1996.

a) Polylysine-DNA complexes with and without copolymer P6INF7:

Polylysine (average chain length 170; Sigma)—DNA was prepared as a stock solution by adding 64 μg pCMVLuc in 800 μl HBS to 256 μg pL in 800 μl HBS and mixing by pipetting. This corresponds to a calculated charge ratio of 6.3. As a positive control, 50 μl each of this suspension of polyplexes were added to column 1 A-F of a 96-well plate and mixed with 100 μl of GVB²⁺ buffer. All other wells contained 50 μl GVB²⁺ buffer. 100 μl were transferred from column 1 to column 2, mixed etc. as described in Plank et al. 1996.

Furthermore, 350 μl each of the polylysine-DNA stock solution were mixed with 35, 70 and 105 nmol (referring to the INF7 moiety) of the polymer P6INF7 and diluted to 1050 μl with GVB²⁺ buffer after 15 min incubation. 150 μl each of the resulting suspension were distributed to column 1, rows A-F, of a 96-well plate. A 1.5-fold dilution series in GVB²⁺ buffer and the rest of the complement activation assay were carried out as described above and in Plank et al. 1996.

The final concentrations of the components in column 1 are 2/3 μg for DNA, 8/3 μg for pL and 0, 5, 10, 15 nmol (referring INF7) for the polymer per 200 μl total volume.

b) Complement Activation by PEI-DNA Complexes With and Without Copolymer Coating:

The assay was carried out as described:

PEI (25 kD, Aldrich)—DNA complexes were prepared by combining equal volumes of a DNA solution (80 μg/ml in 20 mM HEPES pH 7.4) and a PEI solution (83,4 μg/ml in 20 mM HEPES pH 7.4). For the removal of excess unbound PEI, the DNA complexes were centrifuged 3 times for 15 min at 350×g in Centricon-100 filter tubes (Millipore). Between centrifugations, the tubes were filled up to the original volume with 20 mM HEPES pH 7.4. After the final centrifugation step, a DNA complex stock solution corresponding to a DNA concentration of 300 μg/ml was obtained. An aliquot of 182 μl of this solution was diluted to 2520 μl with 20 mM HEPES pH 7.4. Aliquots of 610 μl each (corresponding to 13.2 μg DNA each) were pipetted to solutions of P6YE5C in 277.6 μl 20 mM HEPES pH 7.4. The resulting solutions were adjusted to a salt concentration of 150 mM with 5 M NaCl. 150 μl each of the resulting solutions were transferred to column 1, A-F, of a 96-well plate. The dilution series in GVB²⁺ buffer was carried out as described (Plank et al. 1996).

In the same manner, 610 μl each of a PEI-DNA complex of higher concentration (86 ng DNA per μl) were incubated with 277.6 μl each of solutions of the polymer P3YE5C. The solutions contained 0, 1, 2, 3 charge equivalents of the peptide YE5C relative to the amount of DNA used. After 15 min, 27.45 μl each of 5 M NaCl were added (resulting in a total volume of 915 μl). 150 μl each of the resulting solution were transferred to column 1, rows A to F, of a 96-well plate (this corresponds to 8.6 μg of DNA and 9 μg of PEI each). The dilution series in GVB²⁺ buffer and the remaining assay procedure were carried out as described above for pL-DNA.

The result of the complement activation assay is shown in FIG. 4:

A) Complement activation by polylysine-DNA complexes in the presence and in the absence of the copolymer P6INF7. The CH50 value refers to the particular serum dilution which gives rise to the lysis of 50% of the sheep red blood cells in the setup of the assay. The value CH50ₘₐₓ refers to the particular CH50 value which is obtained with untreated human serum. In the experimental setup described here, human serum was incubated with gene vectors. The CH50 values obtained with serum treated in this manner are lower than CH50ₘₐₓ if gene vectors activate the complement cascade. The data are presented as percentage of CH50ₘₐₓ. The strong complement activation observed with polylysine-DNA complexes can be entirely inhibited by the coating polymer P6INF7.

B) The peptide INF7 itself in free form or polymer-bound, is a weak activator of complement. If incorporated in a polylysine-DNA complex, this complement activation disappears.

C) Complement activation by PEI-DNA complexes (N/P=8) in the presence of the copolymer P3YE5C. The unprotected DNA complex is a strong activator of the complement system. The copolymer P3YE5C reduces the complement activation in dependence of the added amount of copolymer but does not lead to complete protection in the range examined.

D) In contrast, the copolymer P6YE5C completely protects from complement activation even if added in small amounts.

### EXAMPLE 5

### Erythrocyte Lysis Assay

The assay serves the examination of the ability of peptides to lyse natural membranes in a pH-dependent manner.

The erythrocytes used in this assay were obtained as follows: 10 ml of fresh blood were taken from volunteers and diluted immediately into 10 ml of Alsever's solution (Whaley 1985; Plank et al., 1996). Aliquots of 3 ml each were washed 3 times with the corresponding buffer (40 ml each of citrate or HBS; after addition of buffer, shaking, centrifugation at 2500×g and discarding of the supernatant). The concentration of the erythrocytes was determined with an “extinction coefficient” of 2.394×10⁻⁸ ml/cells at 541 nm. For deriving the extinction coefficient, the cell count in an aliquot was determined using a Neubauer chamber followed by measuring the absorbance of this solution at 541 nm upon addition of 1 μl 1% Triton X-100.

Aliquots of INF7 and copolymer-coupled INF7 (P3INF7), respectively, were provided in column 1 of a 96-well plate in 150 μl 10 mM sodium citrate pH 5/150 mM NaCl and in HBS buffer, respectively (usually corresponding to 45 μmol peptide). All other wells were provided with 50 μl buffer each (citrate and HBS, respectively). 100 μl were transferred from column 1 to column 2 using a multichannel pipettor and mixed by pipetting. 100 μl were transferred from column 2 to column 3, and so on. The surplus 100 μl from column 11 were discarded, column 12 contained buffer only. The resulting 1.5-fold dilution series was diluted to 100 μl with 50 μl buffer each (citrate and HBS, respectively). Subsequently, 3×10⁶ human erythrocytes each were added, the plates were sealed with parafilm and shaken at 400 rpm in an incubator shaker (Series 25 Incubator Shaker; New Brunswick Scientific Co.; N.J., U.S.A.) at 37° C. for 1 h. Then, the plates were centrifuged at 2500×g, 150 μl each of the supernatant was transferred into a flat bottom 96-well plate and released hemoglobin was determined at 410 nm using an ELISA plate reader. 100% lysis was determined by addition of 1 ∥l, 1% Triton X-100 to individual wells in column 12 (before transferring to the flat bottom plate). 0% lysis was determined from untreated samples in column 12.

The result of the erythrocyte lysis assay is shown in FIG. 5: Peptide INF7 displays a strong pH-dependent activity. From the synthesis of the copolymer P3INF7, four fractions (decreasing molecular weight from 1 to 4) were isolated upon chromatographic separation (Superdex 75, Pharmacia). Among these, fractions 2 and 3 displayed a higher lysis activity than free peptide INF7. In all cases, the lysis activity was strictly pH-dependent, that is, no lysis at neutral pH (not shown).

### EXAMPLE 6

### Size Determination of DNA Complexes by Dynamic Light Scattering and Electron Microscopy

Preparation of PEI-DNA polyplexes and applying the polymer coating: 40 μg DNA (pCMVLuc) each in 333 μl 20 mM HEPES pH 7.4 were pipetted to 41.7 μg PEI (25 kD, Aldrich) in 333 μl HEPES pH 7.4 and mixed. After 10-15 min incubation, 0, 0.5, 1, 1.5, 2 or 3 charge equivalents (relative to the charge of the applied DNA) of polymers P3YE5C and P6YE5C, respectively, in 333 μl HEPES each were added (or 0, 1, 2, 3 and 5 equivalents in a second experiment). Referring to peptide YE5C, this corresponds to an amount of 0, 152, 303, 455, 606 or 909 pmol polymer per pg DNA. DOTAP/cholesterol-DNA complexes were prepared from DOTAP/cholesterol (1:1 mol/mol) liposomes in 330 μl 20 mM HEPES pH 7.4 and DNA in an equal volume at a charge ratio of 5. The lipoplexes were incubated with 0, 1, 2, 3 and 5 equivalents of the copolymer P3YE5C in 330 μl buffer. The final DNA concentration of the complex was 10 μg/ml.

The size of the DNA complexes was determined on the one hand by dynamic light scattering (Zetamaster 3000, Malvern Instruments) immediately after polymer addition and subsequently at various time points over several hours. On the other hand, the sizes were determined by electron microscopy as described in Erbacher et al., 1998, and Erbacher et al., 1999.

FIG. 6 shows the electron micrographs of PEI-DNA complexes (N/P=8) in the presence of the copolymer P3YE5C.

A) In the presence of one charge equivalent (with respect of the charges of the DNA used) of the copolymer. The particle size is 20 to 30 nm.

B) In the presence of two charge equivalents of the copolymer. The majority of the particles display sizes around 20 nm. These are monomolecular DNA complexes, that is, one plasmid molecule packaged into one particle.

C) In the presence of 1.5 charge equivalents of the copolymer upon addition of BSA to a final concentration of 1 mg/ml and incubation over night. Copolymer-protected DNA complexes remain stable and do not aggregate, in contrast to unprotected PEI-DNA complexes which immediately precipitate under the same conditions (not shown).

### EXAMPLE 7

### Determination of the Zeta Potentials of DNA Complexes

The same samples as in Example 6 were subjected to zeta potential determinations using the Malvern instrument. The parameters of refractive index, viscosity and dielectric constant were set to the values of deionized water, which is valid only as an approximation.

FIG. 7 shows the zeta potentials of PEI- and DOTAP/cholesterol-DNA complexes in dependence of the amount of copolymer P3YE5C added. The zeta potential, a measure of the surface charge of the complexes, drops from highly positive over neutral to slightly negative with increasing amounts of copolymer added. This demonstrates that the copolymer binds to the DNA complexes and neutralizes or shields their electrostatic charges.

### EXAMPLE 8

### Preparation of DNA Complexes and Transfections

For the following examples of cell culture and transfection experiments, the following materials and methods were used, unless stated otherwise:

a) Gene transfer in cell culture in a 96-well plate

Adherent cells are seeded into flat bottom plates at a density of 20,000 to 30,000 cells per well the day prior transfection (dependent on the rate of cell division. The cells should be 70-80% confluent during transfection). Before transfection, the medium is removed by aspiration. For transfection, 150 μl medium is added to the cells, followed by addition of 50 μl of DNA complexes.

b) Composition of the DNA complexes: Preferably, 1 μg DNA/well final concentration. The calculation is carried out for 1.2-fold the amount needed. A volume of 20 μl per component (DNA, PEI, polymer) is used. Finally, 50 μl of DNA complex are used for transfection. Buffer: 20 mM HEPES pH 7.4/150 mM NaCl=HBS. The volumes of buffer used remain constant.

In the case that DNA complexes for 96 individual experiments are required, the calculation is suitably carried out such as if 100 individual experiments were performed, for example:

DNA: 1 μg×100×1.2=120 μg in 20×100 μl HBS=2 ml total volume.

Polyethylenimine (PEI): In order to obtain an N/P ratio of 8, the calculation according to the formula

$$N / P = \frac{( μ gPEI )}{43} \times \frac{330}{( μ gDNA )}$$

$$8 = \frac{( μ gPEI )}{43} \times \frac{330}{( 120 ) ,}$$

shows, that 125.09 μg PEI are required, and this in a total volume of 20×100 μl=2 ml HBS.

Coating polymer: In the case, for example, that coating polymer is to be used for the amount of DNA and PEI indicated above in an amount of 2 charge equivalents (with respect to DNA), the required volume of coating polymer at a concentration of 11.1 μmol/ml, according to the formula

$$μ l ( polymer ) = 1000 \times \frac{( μg DNA )}{330} \times \frac{charge equiv .}{c ( polymer [ μ mol / ml ] )}$$

$$is$$

$$μ l ( polymer ) = 1000 \times \frac{120}{330} \times \frac{2}{11.1} = 65.5 μ l$$

which are diluted to 2 ml with HBS as well.

This is an example for 100 experiments with 1 μg DNA each. Usually, approximately 5 experiments are carried out and, for example, N/P ratios of 4, 5, 6, 7, 8 with 0, 1, 2, 3 charge equivalents each of coating polymer are examined.

c) Mixing of the DNA complexes:

After the preparation of the required dilutions, DNA is added under vortexing to PEI. After 15 min, the coating polymer is added to the preformed PEI-DNA complex, again under vortexing. After further 30 min, 50 μl DNA complex each are added to the cells which are present in 150 μl medium.

The type of vessel used is dependent on the calculated total volum. In the above example, PEI is suitably provided in a 14 ml polypropylene tube (for example Falcon 2059), the other two components are provided in 6 ml tubes (for example, Falcon 2063). For individual experiments in a 96-well plate, the components can also be mixed in a 96-well plate. If the final total volume is 1-1.5 ml, Eppendorf tubes are suitable. A micropipet can be used for mixing instead of vortexing.

Conversion to 3 cm dishes (6-well plate):

For 3 cm dishes, amounts of DNA of 2 to 5 μg are suitably used, with a volume per component, for example, of 100 μl each. The calculation is carried out in analogous manner as above. In a 12-well plate, amounts of ca. 1 μg of DNA per assay are suitably used.

FIG. 8 shows the formulation of DNA complexes in a schematic manner: Preferably, a polycation is first incubated with plasmid DNA, resulting in a positively charged DNA complex (for example, PEI, N/P=8). Then, negatively charged copolymer is added, which electrostatically binds to the preformed complex. The copolymer can be modified with a receptor ligand, as symbolized by asterisks (right).

d) Luciferin Substrate Buffer

As luciferin substrate buffer, a mixture of 60 mM dithiothreitol, 10 mM magnesium sulfate, 1 mM ATP, 30 μM D (-)-luciferin in 25 mM glycyl-glycine buffer pH 7.8 was used.

e) Protein Determination in Cell Lysates

The protein content of the lysates was determined using the Bio-Rad protein assay (Bio-Rad): To 10 μl (or 5 μl) of lysate, 150 μl (or 155 μl) of dist. water and 40 μl Bio-Rad Protein Assay dye concentrate were added per well of a transparent 96-well plate (type _{“}flat bottom”, Nunc, Denmark). The absorbance was determined at 630 nm using the absorbance reader _{“}Biolumin 690” and the computer program _{“}Xperiment” (both Molecular Dynamics, U.S.A.). As a standard curve, concentrations of 50, 33.3, 22.3, 15, 9.9, 6.6, 4.4, 2.9, 2.0, 1.3, 0.9 and 0 ng BSA/μl were measured. Bovine serum albumin (BSA) was purchased as the Bio-Rad Protein Assay Standard II. In this manner, the results could finally be expressed as pg luciferase per mg protein.

### EXAMPLE 9

### Gene Transfer in K562 Cells with PEI(25 kD)-DNA Complexes in the Presence and in the Absence of the Copolymer P3YE5C

K562 cells (ATCC CCL 243) were cultivated at 37° C. in an atmosphere of 5% CO₂ in RPMI-1640 medium supplemented with 10% FCS, 100 units/ml penicillin, 100 μg/ml streptomycin and 2 mM glutamine. The evening prior transfection, desferoxamine was added to a final concentration of 10 μM. Immediately before transfection, the medium was changed. 50,000 cells in 160 μl medium each were plated in the wells of a 96-well plate. Transferrin-PEI (hTf-PEI 25 kD) was prepared by reductive amination essentially as described by Kircheis et al., 1997. A product was obtained having coupled on average 1.7 transferrin molecules per PEI molecule.

In a pilot experiment, a composition of hTf-PEI polyplexes was determined that gives rise to high transfection and that clearly shows an influence of the receptor ligand. hTf-PEI (32.4 μg; amount refers to hTf) in 600 μl HBS was combined with 36 μg PEI (25 kD) in 600 μl HBS. 40 μg of DNA (pCMVLuc) in 600 μl HBS were pipetted to this mixture and mixed. After 15 min, 270 μl of the resulting solution each was added to 90 μl each of solutions of the polymer P3YE5C in HBS and to HBS only, respectively. These solutions contained amounts of polymer which contained 0/0.5/1/1.5/2/3 charge equivalents with respect to the charge of the DNA applied. In analogous manner, DNA complexes without hTf were prepared with the equivalent amount of PEI (40 μg DNA+42 μg PEI+coating polymer). 60 μl each of the resulting mixtures (corresponding to an amount of 1 μg DNA/well) were provided in 5 wells each of a round bottom 96-well plate and 50,000 K562 cells in 160 μl RPMI medium each were added. After 24 h, the cells were sedimented by centrifugation. The supernatant was removed by aspiration, and 100 μl lysis buffer (250 mM Tris pH 7.8; 0.1% Triton X-100) were added. After 15 min incubation and mixing by pipetting, 10 μl sample each were transferred to an opaque plate (Costar) for the luciferase assay in 96-well plate format. The samples were provided with 100 μl luciferin substrate buffer. The measurement of light emission was carried out with a microplate scintillation & luminescence counter _{“}Top Count” (Canberra-Packard, Dreieich). The count time was 12 seconds, the count delay was 10 min, and background counts were automatically substracted. As a standard, 100, 50, 25, 12.6, 6.25, 3.13, 1.57, 0.78, 0.39, 0.2, 0.1, 0.05, 0.025, 0.013, 0.007 and 0 ng luciferase each (Boehringer Mannheim) in 10 μl lysis buffer each (=2-fold dilution series) were measured under the same conditions. A calibration curve was derived from these measurements.

FIG. 9 shows the results of the gene transfer experiments with PEI-DNA complexes (N/P=8) in K562 cells in the presence and in the absence of transferrin as a receptor ligand under the addition of the copolymer P3YE5C. The copolymer does not interfere with gene transfer and even improves it, if a receptor ligand is present in the DNA complex. Shown is the expression of the luciferase reporter gene normalized to the total protein content in the cell extract (averages and standard deviations of triplicates).

### EXAMPLE 10

### Transfection of the Mamma Carcinoma Cell Line MDA-MB435S With Polylysine-DNA Complexes in the Presence and in the Absence of the Coating Polymer P31NF7

MDA-MB435S cells (ATCC?? human mamma carcinoma cell line) were cultivated at 37° C. in an atmosphere of 5% CO₂ in DMEM medium supplemented with 10% FCS, 100 units/ml penicillin, 100 μg/ml streptomycin and 2 mM glutamine. The evening prior transfection, the cells were plated at a density of 20,000 cells per well in flat-bottom 96-well plates.

The DNA complexes were prepared as follows:

Calculation for 1 well: The amount of DNA to be obtained is 1 μg per well, the amount of pL170 is 4 μg in a total volume of 60 μl HBS. The amounts were multiplied by 1.2. The DNA complexes were mixed as specified in the table below, where first DNA was added to polylysine and this mixture was added after 15 min to the polymer P3INF7 and buffer, respectively. The experiments were carried out in triplicates. Sixty μl of DNA complexes each were added to the cells which were covered with 150 μl medium. After 4 h, the medium was changed. After 24 h, the luciferase and protein assays were carried out as described in Example 9 upon washing with PBS and addition of 100 μl lysis buffer.

| | | | | | 7.2 μg |
|---|---|---|---|---|---|
| | | HBS | pL170 | | DNA in |
| Nr. | P3INF7 | μl | μl = μg | HBS | HBS (μl) |
| 1 | 144.6 | 71.4 | 28.8 | 79.2 | 108 |
| | (5 nmol) | | | | |
| 2 | 289.2 | — | 28.8 | 42.6 | 71.4 |
| | (10 nmol) | | | | |
| 3 | — | — | 28.8 | 187.2 | 216 |

FIG. 10 shows the result of the gene transfer experiments into the human mamma carcinoma cell line MDA-MB435S with polylysine-DNA complexes in the presence and in the absence of the copolymer P3INF7. In the absence of the copolymer, no measurable reporter gene expression occurs. The pH-dependent membrane-disrupting and therefore endosomolytic activity of the copolymer gives rise to efficient gene transfer. 5 nmol and 10 nmol P3INF7, respectively, refer to the amount of copolymer-bound peptide INF7 applied.

### EXAMPLE 11

### Lipofection in the Presence of Coating Polymers (FIG. 11)

NIH3T3 cells (ATCC CRL 1658) were cultivated at 37° C. in an atmosphere of 5% CO₂ in DMEM medium supplemented with 10% FCS, 100 units/ml penicillin, 100 μg/ml streptomycin and 2 mM glutamine.

The evening prior transfection, cells were plated at a density of 500,000 cells per well in 6-well plates.

Preparation of DNA complexes:

To 16 μg DNA in 240 μl 20 mM HEPES pH 7.4, a solution of 242 nmol DOTAP/cholesterol liposomes in 240 μl of the same buffer was added. This results in a charge ratio (+/−) of 5. Of the resulting solution, 210 μl were pipetted to 105 μl of a solution containing 6.36 nmol of the polymer P3YE5C (with respect to the peptide moiety YE5C; this corresponds to 3 DNA charge equivalents). For the control experiment, 210 μl DOTAP/cholesterol-DNA were pipetted to 105 μl 20 mM HEPES pH 7.4. 90 μl each of the resulting DNA complexes were added to the cells which were held in 800 μl fresh medium. This corresponds to 2 μg of DNA per well. The experiments were carried out in triplicates.

In the same manner, the experiment was carried out with Lipofectamine™ instead of DOTAP/cholesterol. In this case, an amount of Lipofectamine (DOSPA) was used which gives rise to a charge ratio of 7 (+/−).

30 min after addition of the DNA complexes, 1 ml each of fresh medium was added to the cells, after 3 h additional 2 ml were added. The medium was not changed. 22 h after complex addition, the cells were washed with PBS and lysed in 500 μl lysis buffer. Aliquots of the cell lysate were used for the luciferase assay and for protein content determination.

FIG. 11 shows the result of the lipofection of NIH3T3 cells in the presence and in the absence of the copolymer P3YE5C. Neither the transfection with DOTAP/cholesterol-DNA nor the one with Lipofectamine is significantly reduced (3 charge equivalents of the copolymer. DOTAP/cholesterol-DNA displays a neutral zeta potential at this composition; see FIG. 7).

### EXAMPLE 12

### Transfection of HepG2 Cells with DOTAP/cholesterol-DNA und PEI-DNA in the Presence and in the Absence of P6YE5C

HepG2 cells (ATCC HB 8065) were cultivated at 37° C. in an atmosphere of 5% CO₂ in DMEM medium supplemented with 10% FCS, 100 units/ml penicillin, 100 μg/ml streptomycin and 2 mM glutamine.

Two days prior transfection, the cells were plated in 6-well plates at a density of 500,000 cells per well. The transfection with DOTAP/cholesterol was carried out exactly as described above for NIH3T3 cells, except that this time the polymer P6YE5C was used. Furthermore, 7 μg DNA in 105 μl HEPES buffer were pipetted to 7,3 μg PEI 25 kD dissolved in the same volume. After 15 min incubation, this solution was pipetted to 105 μl of a solution of the polymer P6YE5C containing 3 charge equivalents of YE5C. 90 μl each of this solution were added to the cells. The experiments were carried out in triplicates.

FIG. 12 shows the gene transfer into HepG2 cells in the presence and in the absence of the copolymer P6YE5C. The transfection by DOTAP/cholesterol-DNA is not significantly inhibited. The transfection by PEI-DNA complexes is reduced (3 charge equivalents of the copolymer).

### EXAMPLE 13

### Intravenous Gene Transfer in vivo

a) Control (PEI-DNA, N/P=8):

150 μg DNA (pCLuc) in 337.5 μl 20 mM HEPES pH 7.4 were pipetted to 156.4 μl of PEI (25 kD, Aldrich) in the same volume of HEPES buffer. After 15 min, 75 μl 50% glucose were added. Of this solution, 100 μl each were injected into the tail vein of mice (corresponding to a dose of 20 μg DNA per animal).

b) Control (DOTAP/cholesterol-DNA; charge ratio +/−=5):

DOTAP-cholesterol liposomes were prepared according to a standard protocol (Barron et al., 1998). In this case, liposomes with a molar ratio of DOTAP to cholesterol of 1:1 and a final concentration of 5 mM DOTAP in 5% glucose were prepared. 130 μg DNA in 91.1 μl 20 mM HEPES pH 7.4 were added to 393.5 μl liposome suspension. After 15 min, 65 μl 50% glucose were added. Of this solution, 100 μl each were injected into the tail vein of mice (corresponding to a dose of 20 μg DNA per animal).

c) PEI-DNA (N/P=8) with copolymer coating:

150 μg DNA in 2475 μl were added to 156.4 μg PEI (25 kD) in the same volume under vortexing. After 15 min, 3 charge equivalents (with respect to the charges of the amount of DNA applied) of polymer P3YE5C in 2475 μl HEPES buffer were added under vortexing. After further 30 min, the DNA complexes were concentrated by centrifugation in Centricon 30 tubes to a DNA concentration of 454 μg/ml. This solution was subsequently adjusted to a final concentration of 200 μg DNA per ml and 5% glucose by addition of 50% glucose and 20 mM HEPES pH 7.4. Of this solution, 100 μl each were injected into the tail vein of mice (corresponding to a dose of 20 μg DNA per animal).

d) DOTAP/cholesterol-DNA (5:1) with copolymer coating: 393.9 μl liposome suspension were directly pipetted to a solution of 130 μg DNA in 65.3 μl water. After 15 min, 3 charge equivalents P3YE5C in 216.9 μl HEPES buffer were added and, after further 30 min, 75 μl 5% glucose. Of this solution, 115.5 μl each were injected into the tail vein of mice (corresponding to a dose of 20 μg DNA per animal).

FIG. 13 shows the result of the in vivo gene transfer experiments: PEI-DNA- and DOTAP/cholesterol-DNA complexes with and without bound copolymer P3YE5C (3 charge equivalents) were injected into the tail vein of mice (n=6). The animals were sacrificed 24 h after injection and the reporter gene expression in organs was determined. Each time, the highest activity was measured at the injection sites. With PEI-DNA-copolymer, significant reporter gene expression was found in the lung and in the heart, while gene transfer to the lung by DOTAP/cholesterol-DNA was inhibited by application of the copolymer.

### EXAMPLE 14

### Steric Stabilization of PEI-DNA Complexes

PEI-DNA complexes were prepared exactly as described in Example 6 (PEI-DNA, N/P=8, 0/1.5/3 charge equivalents copolymer P3YE5C and P6YE5C, respectively). The size of the complexes was determined by dynamic light scattering to be 20 to 30 nm. Subsequently, 5 M NaCl were added to a final conentration of 150 mM. PEI-DNA without copolymer aggregated immediately (after 5 min a particle population of >500 nm was measureable, after 15 min the majority of the particles were >1000 nm; the complexes precipitated from the solution over night). In the presence of P3YE5C or P6YE5C, respectively (1.5 or 3 charge equivalents) the particle size remained stable at least over 3 days.

Similarly, the addition of BSA to a final concentration of 1 mg/ml lead to an immediate precipitation of PEI-DNA. In the presence of P3YE5C and P6YE5C, respectively (1.5 charge equivalents and more), the particle size remained constant at least over 24 hr (see also FIG. 6c).

### EXAMPLE 15

### Preparation of Collagen Sponges Loaded With COPROGs (Copolymer-Protected Gene Vectors)

500 μl each of a plasmid DNA solution (coding for luciferase under the control of the CMV promoter; concentration 0.5 mg/ml in water) were added to 500 μl each of a polyethylene imine solution (25 kD; Aldrich; 521 μg/ml in water) using a micropipette and mixed instantly by pipetting. The resulting vector suspension was added to 500 μl of an aqueous solution of the PROCOP (“protective copolymer”) P6YE5C and mixed by instant pipetting. The PROCOP solution contained 2 charge equivalents each of P6YE5C. The charge equivalents refer to the quotient of the (negative) charge in the PROCOP and the negative charge of the DNA. The amount in nmol of PROCOP to be used is calculated according to the formula

$$PROCOP ( n mol ) = \frac{DNA ( μg )}{330} \times CE$$

The amount of PROCOP to be used in microliters is calculated according to

$$PROCOP ( μ l ) = \frac{DNA ( μ g )}{330} \times \frac{CE}{{c}_{PROCOP} ( mM )}$$

where CE are the charge equivalents of PROCOP and C_{PROCOP} is the concentration of the copolymer. The concentration of the polymer is given in terms of the (negative) charges of the (anionic) peptide in the polymer, which in turn are determined by photometric determination of the peptide concentration based on the extinction of the tyrosine in the peptide.

The resulting aqueous vector suspensions were pooled. 3 ml each of vector suspension were applied to 4.5×5 cm Tachotop sponge using a micropipettor (before, the commercially available sponge was cut to pieces of this size, under the sterile bench, weighed and provided in glass petri dishes). After 2 to 3 hours of incubation at room temperature, the petri dishes were briefly subjected to vacuum in a lyophilizer (Hetosicc CD4, Heto), followed by abruptly returning the vacuum chamber to normal pressure (“vacuum loading”). This causes the air bubbles in the sponge to disappear and the sponge to completely soak with liquid. After 4 hours incubation in total, the sponges were dried over night in the petri dishes without prior freezing in the lyophilizer. The sponges were subsequently kept in parafilm-sealed petri dishes at 4° C. until implantation in experimental animals.

### EXAMPLE 16

### Preparation of Collagen Sponges Loaded With Conventional Gene Vectors

(a) Loading with naked plasmid DNA

Under sterile conditions, 500 μg plasmid DNA dissolved in 5 ml 5% glucose were applied to a 4.5×5 cm Tachotop sponge with a pipet. This corresponds to ca. 20 μg DNA per cm². After 24 h incubation at 4° C., the sponge was lyophilized (lyophilizer Hetosicc CD4, Heto, vacuum <10 μbar) and cut to pieces of ca. 1.5×1.5 cm under sterile conditions. Such a piece of sponge consequently corresponds to a load of ca. 45 μg DNA.

Such preparations were used for gene transfer in vitro as described in Example 19.

FIG. 15 shows a low reporter gene expression from the beginning, which becomes undetectable after a short period.

(b) Polyethylene imine/DNA sponges

Pre-treatment of PEI and preparation of DNA complexes:

PEI (25 kD molecular weight) was dissolved in sterile distilled water or in HBS buffer and neutralized by addition of 80 μl concentrated hydrochloric acid per 100 mg PEI. This solution was separated from low molecular weight components with Centricon 30 concentrators (Amicon-Millipore) or by dialysis (molecular weight cut-off 12-14 kD). The concentration of the solution was determined by a ninhydrin assay which quantifies primary amines.

For the formation of DNA complexes, equal volumes of solutions of pDNA and PEI were combined. DNA was added under shaking to the PEI solution. The amount of PEI was chosen to result in a nitrogen-to-phosphate ratio (N/P ratio) of 8:1 and 10:1, respectively. This ratio is the molar ratio of nitrogen atoms in the PEI to the phosphates (=negative charges) of the nucleotides of the DNA.

Calculation:

$$N / P = \frac{( μg PEI )}{43} \times \frac{330}{( μg DNA )}$$

(330=average molecular weight of a nucleotide; 43=MW of the repeating unit of PEI taking into account the primary amines).

Loading of the sponges with PEI-DNA complexes:

5 ml of PEI-DNA complex solutions containing 250 μg, 375 μg or 500 μg DNA and N/P ratios of 8 or 10 were applied to 4.5×5 cm-sized Tachotop or Resorba sponges with a pipet. 250 μg DNA per 4.5×5 cm correspond to 10 μg DNA per cm², 375 μg DNA on 4.5×5 cm correspond to 15 μg DNA per cm² and 500 μg DNA on 4.5×5 cm correspond to 20 μg DNA per cm². After 24 h incubation at 4° C., the preparations were lyophilized and cut to ca. 1.5×1.5 cm pieces under sterile conditions (corresponding to 22,5 μg, 34 μg or 45 μg DNA).

Such preparations were used for gene transfer in vitro such as described in Example 19.

FIG. 15 shows high gene expression. The gene expression was assayed over several weeks. An increase of expression on the sponges was observed (not shown).

(c) Liposome/DNA sponges

Preparation of cationic liposomes from DOTAP powder:

In a silanized screw cap glass tube, a 5 mM DOTAP in chloroform solution was prepared. The chloroform was removed by rotary evaporation (Rotavapor-R, Büchi, Switzerland) so that a uniform lipid film was formed on the inner surface of the tube. The rotary evaporator was ventilated with argon gas in order to exclude oxygen. The tubes were subjected to the vacuum of the lyophilizer over night. The lipid film was subsequently rehydrated with 15 ml of a 5% glucose solution, first, under vortexing for 30 seconds, and then under treatment with ultra sound (Sonicator: Sonorex RK 510 H, Bandelin) for 30 min which resulted in the formation of a stable liposome suspension.

Preparation of DOTAP lipoplexes:

For a 4.5×5 cm sponge, 222 μg DNA are required in order to obtain 20 μg DNA per 1.5×1.5 cm. The charge ratio (+/−) should be 5:1, where the positive charges originate from DOTAP and the negative charges from the DNA. 222 μg DNA correspond to 0,67 μmol negative charges. In a polystyrene tube, 3.35 μmol DOTAP liposomes were diluted to a volume of 2.5 ml with 5% glucose solution. To this, 222 μg DNA, also in 2.5 ml glucose solution, were added under slight shaking.

Application of DOTAP lipoplexes to the sponge:

5 ml of the above prepared liposome/DNA solution were evenly distributed on a 4.5×5 cm Tachotop sponge under sterile conditions using a pipet. After 24 h incubation at 4° C., the sponge was lyophilized and subsequently cut to pieces of 1.5×1.5 cm.

(d) DNA/DOTAP sponges

500 μg DNA (pCMVLuc) in 5% glucose solution were pipetted on a 4.5×5 cm Tachotop sponge under sterile condtions, incubated at 4° C. for 24 h and subsequently lyophilized. This corresponds to 20 μg DNA per 1×1 cm and 45 μg DNA per 1.5×1.5 cm, respectively.

In a pilot experiment, it was demonstrated by loading of a 0,01% methyl violet-chloroform solution to a collagen sponge, that the entire sponge structure was evenly moistened by the solution. From this it was concluded that this should be possible as well for a lipid solution in chloroform. The desired charge ratio should be 5. For 500 μg DNA, this requires an amount of 7.6 μmol DOTAP. Accordingly, 5 ml of a 1 mg/ml DOTAP solution in chloroform were loaded on the sponge. Subsequently, the sponge was incubated at −20° C. and then over night at room temperature (in order to allow the chloroform to evaporate). The sponge was cut to ca. 1.5×1.5 cm pieces under sterile conditions.

(e) DOTAP/DNA sponges

The desired charge ratio was again 5:1. 5 ml of a 1 mg/ml DOTAP solution in chloroform were applied to 4.5×5 cm Tachotop, Tissu Vies and Resorba sponge, respectively, using a pipet and incubated for ca. 1 h at −20° C. The chloroform evaporated over night at room temperature. 500 μg DNA (pCMVLuc) in 5 ml 5% glucose solution were applied per 4.5×5 cm sponge with a pipet, incubated for 24 h at 4° C. and subsequently lyophilized. This corresponds to 20 μg DNA per cm².

(f) DOTAP-cholesterol/DNA sponges

The desired charge ratio (+/−) was again 5:1, the desired DNA load was 20 μg per cm². Hence, 5 mg of DOTAP and 2.95 mg cholesterol (this is 305 nmol each) were dissolved in 2.5 ml chloroform each and subsequently combined.

This solution was applied to a 4.5×5 cm Tachotop sponge with a pipet, incubated for 1 h at −20° C. followed by evaporation of the chloroform at room temperature. 500 μg DNA (pCMVLuc) in 5 ml 5% glucose solution were applied to the 4.5×5 cm sponge with a pipet, incubated for 24 h at 4° C. and lyophilized. Subsequently, the sponge was cut to 1.5×1.5 cm pieces.

Variant:

180 ml of a DOTAP:cholesterol =1:0.9 solution were prepared at a total lipid concentration of 2 mM in chloroform. Tachotop sponges (Nycomed) were cut in half (=4.5×5 cm) and immersed in 30 ml each of this solution in 50 ml polypropylene screw cap tubes followed by 2 h incubation on a shaker incubator. Intermittently, the tubes were slightly evacuated for a short time with the cap opened (“vacuum loading”) in the lyophilizer, such that the sponges got entirely soaked with the chloroform solution. The sponges were finally transferred from the chloroform bath into glass petri dishes and dried over night under vacuum. 500 μg DNA (pCMVLuc) in 5 ml 5% glucose solution were trickled on 4.5×5 cm sponge each, incubated for 4 h at room temperature and lyophilized. The sponge was subsequently cut to pieces of 1.5×1.5 cm. Such preparations were used for gene transfer in vitro such as described in Example 19.

Initially, high expression which fades rapidly is observed in cells in the culture dish. In contrast, expression on the sponge remains constant and persists over a long time period. FIG. 15

(g) DNA/PEI-SH-SPDP sponges

(i) Covalent coupling of PEI to the sponges:

0.5 ml of a 15.5 mM SPDP solution in abs. ethanol were added to 2 ml 0.1 M HEPES pH =7.9, mixed, applied to 4.5×5 cm Tachotop sponges with a pipet and incubated over night at 37° C. The amino groups of lysines in the collagen react in a nucleophile substitution reaction with the carboxyl groups of the activated esters in SPDP.

Unbound SPDP was washed out quantitatively with distilled water (in 14 ml Falcon tubes; Becton Dickinson, USA) until no more absorbtion between 200 and 400 nm could be photometrically determined in the supernatants. Subsequently, the sponges were lyophilized and cut to ca. 1.5×1.5 cm pieces. The sponge pieces were weighed (with a MC 1 balance from Sartorius, Göttingen). For the determination of coupled SPDP, a sponge piece was incubated with 2 ml HBS and 3 μl β-mercaptoethanol. The thiopyridone released during this procedure was determined photometrically at 342 nm (ε=8080 l/mol). The substitution is calculated according to:

$$Substitution ( n mol / mg ) = \frac{{E}_{342} \times {Vol}_{( ml )} \times {10}^{6}}{{ϵ}_{( I / mol )} \times {Weight}_{( mg )}}$$

On average, the substitution was approximately 20 nmol SPDP/mg collagen. But also sponges with 0.45 nmol SPDP/mg collagen were prepared.

(ii) Derivatization of PEI with iminothiolane (Traut's reagent).

In order to couple PEI covalently to the SPDP and with this to the sponge via a disulfide bridge, a thiol group must be introduced into PEI. This was carried out by coupling of 2-iminothiolane to PEI. PEI was mixed with a twofold excess of iminothiolane while rinsing with argon. 1/15 volume 1 M HEPES pH=7,9 was added. Subsequently the reaction continued at room temperature for ca. 20 min. Excess reagent was removed by repeated centrifugation in Centricon 30 tubes. The free thiol groups on the PEI were determined with Elman's reagent.

(iii) Coupling of the PEI-iminothiolane derivative to SPDP-collagen

A five- to ten-fold excess of PEI (with respect to the ratio of free thiol groups on the PEI over thiopyridyl groups on the sponge) was added to the SPDP-sponge pieces. After 7 days at room temperature, the reaction was complete. This was determined by photometric determination of the absorbance at 342 nm. 100% of the SPDP on the sponge had reacted with PEI-SH.

The amount of coupled polyethylene imine is calculatied according to:

$$Substitution ( n mol / mg ) = \frac{{E}_{342} \times {Vol}_{( ml )} \times {10}^{6}}{{ϵ}_{( I / mol )} \times {Weight}_{( mg )}}$$

The sponges were rinsed with water until no more absorbance at 342 nm could be detected in the supernatant. Then the sponges were lyophilized.

(iv) Application of DNA to PEI-SH-SPDP-sponges

20 μg DNA (pCMVLuc) in 500 μl 5% glucose solution were loaded with a pipet per 1.5×1.5 cm sponge piece, incubated for 24 h at 4° C. and lyophilized.

(v) DNA/peptide-SPDP-sponges

Sponges were loaded with SPDP as described. An average substitution of 20 nmol SPDP/mg collagen was obtained. But also sponges with 12.8 nmol SPDP/mg collagen were prepared. Peptide SFO7-SH of the sequence (KKKK)₂KGGC was applied to the sponge in twofold molar excess over the SPDP groups in 300 μl 0.1 M HEPES pH=7.9. The reaction was carried out in a 14 ml Falcon tube where the air space of the tube was shortly rinsed with argon. After 2 days at room temperature, the reaction was 60% complete. This was determined by the absorbtion of the supernatant at 342 nm (determination of released thiopyridone). The calculation of the amount of coupled peptide was carried out as described for PEI.

The peptide-SPDP-sponges were washed with distilled water until no more absorbance at 280 nm was measureable. Subsequently, the sponges were lyophilized.

(vi) Application of DNA to peptide-SPDP-sponges

20 μg DNA (pCMVLuc) in 500 μl 5% glucose solution were loaded per 1.5×1.5 cm sponge piece with a pipet, incubated for 24 h at 4° C. and lyophilized.

### EXAMPLE 17

### Subcutaneous Implantation in Wistar Rats and Determination of Reporter Gene Expression

(a) Experimental animals

Seven two months old male Wistar rats (Charles River Deutschland GmbH, Sulzfeld) with a body weight of 300-400g were used as experimental animals. The rats are held in groups in Makrolon type 4 cages at a maximum occupancy of 5 animals. As sole nutrition, the animals have at their disposal pellets of Altromin 1324, Diet for Rats and Mice (Altromin, Lage/Lippe, Germany) and water ad libitum. The animals are held on sterilized, dust-free granules of softwood which is changed twice weekly. According to the regulations of experimental animal keeping, the animals are accomodated in specialized rooms of an animal facility for conventional animal keeping at a room temperature o 20-25° C. with constant air ventilation. The relative humidity is 60%-70%. Illumination: 12 hours phases each of a light-dark cycle. The light intensity is 50-100 lux. The animals are held for at least 2 weeks prior to experimentation in the animal facility of the Institute and are not set empty before surgery.

(b) Sponge implantation

(i) Materials:

Anesthesia apparatus (MDS Matrx anesthesia apparatus) with Isofluran (Abbot GmbH, Wiesbaden, Germany): This is a cyclic system with a ventilator which disposes of stale air and provides fresh air. The advantages are constant inhalation at surgical tolerance without the need of injected narcotics and the opportunity of fine-tuning of the depth of anesthesia. No pre-medication is required and the animal regains conscience within a few minutes post anesthesia.

Transparent acrylic glass whole-body chamber with a lid

Head chamber

Heating pad (set to level 2, ˜38° C.)

Green cover cloth for the surgical desk and the rat, respectively

Clippers

Skin disinfectant (Cutasept®F, Bode Chemie, Hamburg, Germany)

Bepanthen®Roche eye ointment (Hoffmann-La Roche AG, Grenzach-Wyhlen, Germany)

Water-resistant permanent pen for labeling the rats

sterile disposable gloves

sterile surgical set of instruments consisting of:

1 anatomical forceps

1 surgical forceps

1 Lexer-Scissors with a pointed on a blunted blade

1 convex Metzenbaum-Scissors (pointed/pointed)

1 needle holder gauze swab

Surgical suture: monofile, blue, 45 cm long, 4/0 Prolene® suture with pointed sealed-on needle

sterile disposable No. 15 scalpel

14 numbered and weighed sponges per experimental group (7 animals)

(ii) Surgery:

The animals are moved into the surgery room ca. 15 min prior surgery, in order to let them adapt to the environment. The whole-body chamber which is connected with the anesthesia device is flooded with oxygen/4% Isofluran (350 cm³/min) approx. 2 min prior initializing anesthesia. This is done to achieve the corresponding concentration of the narcotic which will warrant the fastest and with this more gentle initialization of anesthesia possible (short excitation stage). The rat is placed into the whole-body chamber, and the initial concentration of the inhalation gases is held constant until—after 1 to 2 minutes—the righting reflex is lost (rat remains on its back) and anesthesia stage 111.1-2 is reached. The rat is taken out of the chamber, put in ventral position and provided with the head chamber. Once the animal has reached anesthesia stage 111.2, the stage of surgical tolerance (the pedal withdraw reflex should be negative), the Isofluran supply is reduced to 1.5%. A greasing eye ointment is applied to both eyes in order to prevent drying-up of the cornea due to the loss of the palpepral reflex. In the regio lumbalis' (in the dorsal area between last rib and hind extremity), a 7×2 cm area is shaved using the clippers followed by cleansing and disinfecting the skin areas with a Cutasept-sprayed gauze swab. The skin is grasped ca. 2 cm from the median with surgical forceps and a 1 cm incision is made with a scalpel in dorso-ventral direction. Using Metzenbaum scissors, the skin incision is extended in a blunted manner and the subcutaneous tissue is undermined ca. 3 cm in cranial direction. The cranial periphery of the wound is held open with surgical forceps and the prepared sponge is advanced as far as possible in cranial direction into the undermined tissue. The incision is closed with a U-shaped clamp. The same procedure is repeated on the left side (see 5.-7.). The Isofluran supply is shut down while the O₂ perfusion is continued. After re-appearance of the swallowing reflex, 0.1 ml of Novalgin® (active substance: Metamizole-Sodium;. Hoechst AG, Frankfurt, Germany) is orally applied to the animal as a non-steroidal analgetic. The animal is placed into a single-occupancy cage until full recovery of conscience and is returned to its cage after approx. 1 hour.

(c) Sponge recovery

(i) Materials:

Anesthesia apparatus (MDS Matrx anesthesia apparatus) with Isofluran (Abbot GmbH, Wiesbaden, Germany)

Transparent acrylic glass whole-body chamber with a lid

Head chamber

sterile disposable gloves

sterile surgical set of instruments (see above)

1000 ml isotonic sodium chloride infusion solution (Delta-Pharma GmbH, Pfullingen, Germany), provided with 50,000 I.E. heparin (2×5 ml injection solution Heparin-Sodium of 25,000 I.E. each from ratiopharm® GmbH, Ulm/Donautal, Germany)

Infusion tube

Butterfly cannula 19 G

The animals are perfused prior to sponge recovery in order to obtain as far as possible blood-drained tissue. This aims at reducing the number of factors potentially interfering with the subsequent luciferase assay (for example hemoglobin) to a minimum. 2 ml screw cap homogenization tubes (disposable/conical 2.0 ml screw cap tube with cap, VWR scientific products, West Chester, U.S.A.) are filled up to the 0.3 ml mark with large homogenization beads (Zirconia Beads, 2.5 mm Dia, Biospec Products, Inc., Bartlesville, U.S.A.) and with 750 μl each of lysis buffer for animal experiments (10 ml 5×Reporter Lysis Buffer; Promega Corporation, Madison, U.S.A.; +40 ml dd H₂O+1 tablet Protease-Inhibitor Complete™; Boehringer Mannheim GmbH, Germany). These tubes will receive the recovered sponges.

(ii) Procedure:

The rat is pre-treated and anasthesized as described under Sponge Implantation 1.-2. The animal is placed in dorsal position. The abdominal cavity is opened with scissors in a median incision extending from pre-umbillical to the manubrium sterni. Relief incisions are made to the right and the left of the ultimate rib. The vena cava caudalis is exposed and a butterfly cannula is inserted in caudal position into the junction with the venae renales. The infusion solution is connected. After infusion of ca. 5 ml, the vena cava caudalis is opened with a scalpel in caudal position of the insertion point. The animal is perfused with 100-150 ml infusion solution or desanguinized until a distinct de- coloration of the liver is evident. The rat is placed in ventral position. Incision of the skin in the median region using a scalpel, extending from the lumbal region to ca. 7 cm in cranial direction; relief incisions to the left and the right caudal to the implantation wounds. The sponges are largely dissected free with scissors and scalpel, respectively, and removed together with surrounding tissue (connective tissue and a ca. 1 cm portion of the musculus longissimus dorsi). Each recovered sponge (with surrounding tissue) is washed with 1×PBS buffer; sponge and tissue are now separated and are transferred to the labeled homogenization tubes previously prepared. The filled tubes are then placed on ice and processed immediately, if possible.

(iii) Processing of samples:

The samples, which are to be kept on ice continuously, are homogenized using a Mini Bead Beater® (Biospec Products, Inc., Bartlesville, U.S.A.) for 3×20 seconds followed by centrifugation at 14,000 rpm for 10 min at 4° C.

Luciferase assay:

Per tube, 20 μl of supernatant are removed and transferred into the wells of a Costar® 96-well-plate (opaque plate—solid black 96 well, Corning Costar Corporation, Cambridge, U.S.A.). Per well, 100 μl luciferase buffer (Promega Luciferase Assay System, Promega Corporation, Madison, U.S.A.) are added and measured for 12 sec with a count delay of 1 min.

The results of the above-described in vivo experiments are presented in the following table.

| PEI-DNA N/P = 8 | | | | DOTAP/cholesterol-DNA | |
|---|---|---|---|---|---|
| + 2 equiv. P6YE5C | | Naked DNA | | Charge ratio 5:1 | |
| Left | Right | Left | Right | Left | Right |
| sponge | sponge | sponge | sponge | sponge | sponge |
| 214.30 | 80.44 | 0 | 0 | 0 | 0 |
| 212.17 | 90.53 | 0 | 0 | 0 | 0 |
| 40.69 | 45.67 | 0 | 0 | 0 | 0 |
| 169.50 | 91.69 | 0 | 0 | 0 | 0 |
| 18.90 | 16.51 | 0 | 0 | 0 | 0 |
| 475.44 | 72.68 | 0 | 0 | 0 | 0 |
| 0.00 | 0.00 | 0 | 0 | 0 | 0 |

The table shows gene transfer in vivo upon subcutaneous implantation of sponge preparations. The sponges were prepared as described in Examples 15 and 16, respectively, and were implanted subcutaneously in Wistar rats as discribed in Example 16. The gene expression first of all was determined after 3 days. Only collagen sponges loaded with PEI-DNA complexes coated with a copolymer of the invention give rise to detectable reporter gene expression under this experimental setup (numbers are fg luciferase/mg protein).

### EXAMPLE 17

### Release of Radioactive-labeled DNA from Various Collagen Sponge—Vector Preparations

(a) Radioactive Labeling of Plasmid DNA by Nick Translation

The nick translation kit from Amersham (# N5500) was used. Per labeling reaction, 1 μg DNA (pCMVβGal) was used. The protocol of the manufacturer was changed such that the reaction time was 15 min at 15° C. instead of the 2 h at 15° C. suggested for linear DNA. [α-³²P] dATP with a specific activity of 3000 Ci/mmol (Amersham, Freiburg) was used as the nucleoside triphosphate. The separation of unincorporated [β-³²P] dATP was carried out according to the principle of gel filtration and the protocol of the manufacturer with “Nuc Trap Probe Purification Columns” and the acrylic glass-shielded fixation apparatus “Push Column Beta Shield Device” (both from Stratagene, Heidelberg). The resulting plasmid was examined by agarose gel electrophoresis (1% agarose gel, 100 V, 35 min, ethidium bromide staining). It was loaded mixed with unlabeled plasmid and visualized under UV light and by autoradiography after electrophoresis and drying of the gel. This allows assessing the size and the relative fraction of the plasmid fragments formed during the nick labeling. In order to separate the radioactive-labeled DNA from enzymes, the “Promega Wizard TM PCR Preps DNA Purification System” (Promega, U.S.A.) was used with a minor modification of the manufacturer's protocol concerning the equipment.

(b) Preparation of chemically modified sponges with radioactive-labeled DNA

(i) DOTAP/DNA-Tachotop sponges

Tachotop sponges were cut to pieces of ca. 1.5×1.5 cm and weighed. The average weight was 5 mg. Then, 450 μl of a 1 mg/ml DOTAP in chloroform solution were applied to the sponge with a pipet, incubated for 1 h at −20° C., followed by evaporation of the chloroform at room temperature and weighing of the sponges. These DOTAP sponges were placed in the wells of a 6-well plate. A mixture of 20 μg (in one instance also 40 μg) unlabeled plasmid and 10 μl and 30 μl, respectively, of the product of the radioactive labeling per 5 mg sponge in a total volume of 200 μl 5% glucose solution were applied to the sponge using a pipet, incubated at 4° C. for 2-24 h and lyophilized.

(ii) DNA-Tachotop sponges

Method 1: Tachotop sponges were cut to pieces of ca. 1.5×1.5 cm and weighed. The sponges were placed in the wells of a 6-well plate. 20 μg unlabeled plasmid-DNA per 5 mg sponge and 10 or 30 μl radioactively labeled DNA (in a total volume of 200 μl 5% glucose solution) were loaded with a pipet, incubated at 4° C. for 2-24 h and lyophilized.

Method 2:

On a 4.5×5 cm Tachotop sponge, 500 μg unlabeled plasmid DNA and 122.1 μl radioactive-labeled DNA (in a total of 2 ml 5% glucose solution) were loaded with a pipet, incubated for 12 h at 4° C. and lyophilized. The sponge was cut to pieces of 1.5×1.5 cm, and each piece was weighed. In order to determine the fraction of the DNA applied that remained in the cell culture dish upon lyophilization during sponge preparation, the lid and the bottom of the plate were rinsed with 2 ml 10×SDS each of which 40 μl aliquots were measured.

(iii) DOTAP/cholesterol/DNA-Tachotop sponges

The desired charge ratio (+/−) was again 5:1, the desired substitution with DNA was 20 μg per cm². Hence, 5 mg DOTAP and 2.95 mg cholesterol (which is 305 nmol each) were dissolved in 2.5 ml chloroform each and subsequently combined. This solution was applied to a 4.5×5 cm Tachotop sponge with a pipet, incubated for 1 h at −20° C. followed by evaporation of the chloroform at room temperature. 500 μg unlabeled plasmid DNA and 122.1 μl radioactive-labeled DNA (in a total volume of 2 ml 5% glucose solution) were loaded on the 4.5×5 cm sponge with a pipet, incubated for 24 h at 4° C. and lyophilized. The sponge was cut to pieces of 1.5×1.5 cm, and each piece was weighed. In order to determine the fraction of the DNA applied that remained in the cell culture dish upon lyophilization during sponge preparation, the lid and the bottom of the plate were rinsed with 2 ml 10×SDS each of which 40 μl aliquots were measured.

(iv) Polyethylene imine/DNA-Tachotop sponges

On 4.5×5 cm Tachotop sponges, 2 ml PEI/DNA complex solutions (with 500 μg unlabeled plasmid DNA and 122.1 μl radioactive-labeled DNA at an N/P ratio of 6) were loaded with a pipet. The 500 μg DNA per 4.5×5 cm correspond to 20 μg DNA per cm². After 24 h incubation at 4° C., the preparations were lyophilized, cut to ca. 1.5×1.5 cm pieces, and each piece was weighed. In order to determine the fraction of the DNA applied that remained in the cell culture dish upon lyophilization during sponge preparation, the lid and the bottom of the plate were rinsed with 2 ml 10×SDS each of which 40 μl aliquots were measured.

(v) DNA-peptide-SPDP sponges were prepared as described in Example 16 with the one exception that the DNA component contained radioactive-labeled DNA as described above.

(vi) Copolymer-protected polyethylene imine/DNA-Tachotop sponges These sponges were prepared as described in Example 15 with the one exception that the plasmid DNA solution additionally contains radioactive-labeled DNA.

(c) Determination of the Time-Dependent Release of Radioactive-Labeled DNA From the Sponges

The various sponge preparations were provided with 1 ml PBS each in silanized glass tubes (16×100 mm culture tubes with screw caps made from AR glass, Brand, Germany). The tubes were briefly centrifuged at 3,000 rpm (centrifuge: Megafuge 2.0 R, Heraeus, Munich) and then shaken at 37° C. in a water bath shaker at 80 or 120 rpm. After 1 h, 1 day, 3 days and subsequently every 3 days, the amount of radioactive DNA in the supernatant was determined. For this purpose, the tubes were centrifuged at 3,000 rpm and briefly vortexed. 40 μl of supernatant were removed and replaced with 40 μl of PBS. The samples were mixed with 160 μl Microscint 20 high efficiency LSC-cocktail (Packard, U.S.A.) in the wells of a white 96-well opaque plate (type _{“}flat bottom, non-treated”, Costar, U.S.A.) and counted using a Top Count instrument (Canberra-Packard, U.S.A.) under automatic correction for the half-life. The count time was 5 min, the count delay was 10 min, and the average of 3 measurements was formed. As a reference, 2 μl of the labeled plasmid DNA were measured. The measured concentration of DNA (cpm/ml) was corrected for the samples already taken before (amounts removed before were summed up and added to the measured value). In order to determine how much of the DNA applied remained in the cell culture dishes upon lyophilization during sponge preparation, the dishes were rinsed with 500 μl PBS of which aliquots of 40 μl were measured. At the end of a series of measurements (for example after 30 days of incubation), the sponges were treated with a 1% SDS solution in order to determine whether 100% of the applied dose could be recovered. For this purpose, the sponges were transferred to fresh Falcon tubes, 1 ml 1% SDS were added and the samples were incubated for 1 day with repeated vigorous shaking. Then, 40 μl of supernatants were removed, mixed with 160 μl Microscint 20 in the wells of a white 96-well opaque plate and the radioactivity was counted using the Top Count instrument.

The results are shown in FIG. 14.

Sponges loaded with naked DNA release 50% of the applied dose within 1 hour, followed by an approximately linear protracted release. In contrast, vector-loaded sponges display little initial release of not more than 5% followed by a long-time minor release per time unit. This indicates efficient binding of the examined vectors to the collagen matrix.

(d) Agarose Gel Electrophoresis for the Characterization of Released DNA

After 5 and 30 days, respectively, of shaking the DOTAP/DNA sponges in 1 ml PBS, 20 μl each of the supernatant were subjected to electrophoresis for 35 min at 100 V on a small ethidium bromide-stained 1% agarose gel. As a control, 1 μg of unlabeled plasmid DNA and liposome-DNA complexes (charge ratio 5:1) were loaded on the gel. The gel was photographed under UV light, subsequently dried and exposed on a X-ray film.

### EXAMPLE 19

### Transfection of NIH 3T3 Cells Bylon Vector-Loaded Collagen Sponges in vitro

In cell culture plates (6-well plates of the company TPP), ca. 50,000 to 400,000 trypsinized NIH 3T3 mouse fibroblasts (adherent) per well are seeded in 4 ml DMEM medium (Dulbecco's Modified Eagles Medium) supplemented with antibiotics (500 units penicillin, 50 mg streptomycin/500 ml) and 10% fetal calf serum as well as 1.028 g/l N-acetyl-L-alanyl-L-glutamine. The cells are incubated for 1 to 2 days in an atmosphere (air) of 5% carbon dioxide at 37° C. One collagen sponge (1.5×1.5 cm) prepared according to Examples 15 and 16, respectively, is placed into each of an appropriate number of wells 1 to 2 days after seeding of the cells and is incubated for ca. 3 days at 37° C. in an atmosphere of 5% carbon dioxide. The first measurement of luciferase expression is carried out in a period of 1 to 3 days. For this purpose, the wells with the adherent cells are washed 3 times with phosphate buffer solution (PBS) after removal of the collagen sponges and are subsequently treated with 500 μl lysis buffer (0,1% Triton in 250 mM Tris, pH=7,8). Subsequently, the luciferase activity is determined as described below.

In order to prove the protracted effect, the removed collagen sponges are again placed into fresh wells with seeded cells and are incubated for ca. 3 days at 37° C. in an atmosphere of 5% carbon dioxide. After that, the collagen sponges are removed from the wells, the adherent cells are washed and treated with lysis buffer as described above, followed by the determination of the luciferase activity as described below. This procedure is repeated any number of times dependent on how many individual setups were chosen to start with. In this manner, it can be determined over a period of at least 6 weeks to which extent the collagen sponges prepared according to A) are able to transfect, i.e. leading to the expression of luciferase activity in the cells.

Luciferase assay:

Colonized collagen sponges were removed from the tissue culture dishes and washed with PBS. In the same manner, the cells in the tissue culture dishes were washed with PBS. Cells that were eventually detached from the sponges during washing were pelleted from the washing solution by centrifugation and separately examined for luciferase expression. The values derived from this were added to the luciferase expression on the sponge. Cells on the sponges were lysed by addition of 1 ml lysis buffer. Cells in the wells were lysed by addition of 500 μl lysis buffer. 10 to 50 μl cell lysates were mixed with 100 μl each of luciferin substrate buffer in a black 96-well plate. The measurement of the resulting light emission was carried out using a Microplate Scintillation & Luminescence counter “Top Count” (Canberra-Packard, Dreieich). The count time was 12 seconds, the count delay was 10 min and background values were automatically subtracted. As a standard, 100, 50, 25, 12.6, 6.25, 3.13, 1.57, 0.78, 0.39, 0.2, 0.1, 0.05, 0.025, 0.013, 0.007 and 0 ng luciferase in 50 μl lysis buffer each (=2-fold dilution series) were measured under the same conditions, and from this a calibration curve was derived.

Buffers:

(a) Lysis buffer

0.1% Triton X-100 in 250 mM Tris pH 7.8

Luciferin substrate buffer

60 mM dithiothreitol, 10 mM magnesium sulfate, 1 mM ATP, 30 μM D-luciferin, in 25 mM glycyl-glycine buffer pH 7.8.

(b) HEPES-buffered saline (HBS)

20 mM HEPES, pH 7.3; 150 mM sodium chloride

Protein content determination in cell lysates:

The protein content of the lysates was determined using the Bio-Rad protein assay (Bio-Rad, Munich): To 10 μl (or 5 μl) of the lysate, 150 μl (or 155 μl) of dist. water and 40 μl Bio-Rad Protein Assay dye concentrate were added to a well of a transparent 96-well plate (type “flat bottom”, Nunc, Denmark). The absorbtion at 630 nm was read using the absorbance reader “Biolumin 690” and the computer program _{“}Xperiment” (both Molecular Dynamics, U.S.A.). For a calibration curve, concentrations 50, 33.3, 22.3, 15, 9.9, 6.6, 4.4, 2.9, 2.0, 1.3, 0.9 and 0 ng BSA/μl were measured. Bovine serum albumin (BSA) was purchased as Bio-Rad Protein Assay Standard II. In this manner, results can be given as μg luciferase/mg protein.

The results of the in vitro experiments are shown in FIG. 15.

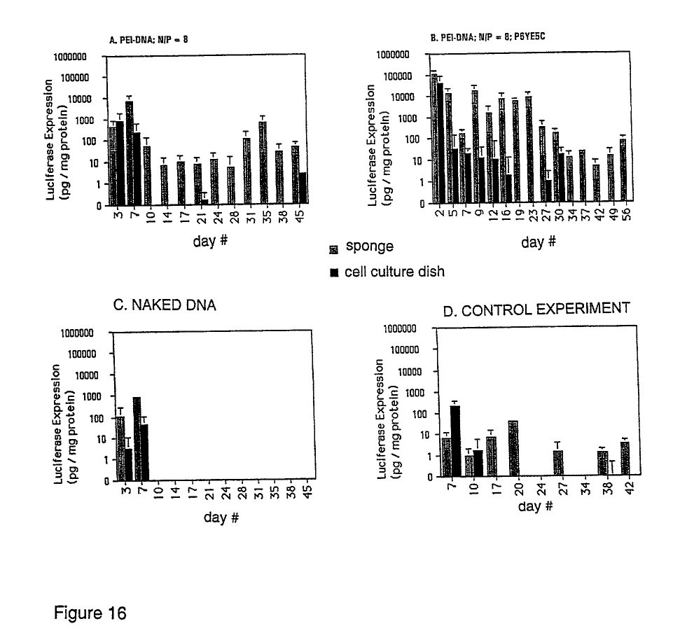

The results of a continuation of the experiments are shown in FIG. 16 A for PEI-DNA and in FIG. 16C for naked DNA. An analogous experiment for sponges loaded with a copolymer-protected gene vector is shown in FIG. 16B. FIG. 16D shows the results of a control experiment. For this purpose, NIH 3T3 fibroblasts were seeded at a density of 450,000 cells per well in a 6-well plate the day prior transfection (e.g. on day 1). Shortly before transfection, the medium was replaced with 1.5 ml fresh medium. The DNA complexes were added in a total volume of 500 μl (day 2), followed by 4 hours of incubation and a medium change. On day 3, an untreated 1.5×1.5 cm-sized piece of collagen sponge was added to each well. On day 6, fresh cells were seeded in fresh 6-well plates (450,000 cells per well). On day 7, all except 3 sponges were moved to these wells. Three sponges and the wells from which all the sponges were taken were subjected to the luciferase assay. On day 10, all except 3 sponges were moved to empty wells and were further incubated in 2 ml medium. Three sponges and all the wells from wich the sponges were moved were subjected to the luciferase assay. At the subsequent time points indicated in FIG. 16D, 3 sponges each and the cells that had sedimented to the bottom of the wells were analyzed for luciferase expression.

FIG. 16D shows that the luciferase expression is initially high but then drops rapidly or is no longer measureable at all. This means that in the other cases (FIG. 15 and 16A-C) the significantly high luciferase expression is to be attributed to continuous de novo transfection by immobilized vectors. Hence, one is not dealing with a whatever selection of initially transfected cells. If this were so, the luciferase expression in the control experiment had to persist on similarly high levels as in the other experiments.

### Literature

Balkenhohl, F., von dem Bussche-Hünnefeld, C., Lansky, A. and Zechel, C. (1996). Angew. Chem. 108: 2436-2488.

Barron, L. G., Meyer, K. B. and Szoka, F. C. J. (1998). Hum. Gene Ther. 10(9(3)): 315-23.

Boussif, O., Lezoualch, F., Zanta, M. A., Mergny, M. D., Scherman, D., Demeneix, B., & Behr, J. P. (1995) Proc. Natl. Acad. Sci. U.S.A. 92, 7297-7301.

Brocchini, S., James, K., Tangpasuthadol, V. and Kohn, J. (1997). J. Am. Chem. Soc. 119(19): 4553-4554.

Coombes, A. G. A., Tasker, S., Lindblad, M., Holmgren, J., Hoste, K., Toncheva, V., Schacht, E., Davies, M. C., Ilium, L. and Davis, S. S. (1997). Biomaterials 18(17): 1153-1161.

Erbacher, P., Remy, J. -S. and Behr, J. -P. (1999). Gene Ther. 6(1):138-45

Erbacher, P., Zou, S. M., Bettinger, T., Steffan, A. M. and Remy, J. S. (1998). Pharm. Res. 15(9): 1332-1339.

Felix, A., M., Heimer, E., P., Lambros, T., J., Tzongraki, C., Meienhofer, J. (1978). J. Org. Chem. 43, 4194-4196.

Ferrari, S., Moro, E., Pettenazzo, A., Behr, J. P., Zacchello, F., & Scarpa, M. (1997) Gene Ther. 4, 1100-1106.

Fields, G., B., Noble, R., L. (1990). Int. J. Peptide Protein Res. 35, 161-214.

Fields, G., B., et al., (1991). Pept. Res. 4, 88.

Gill S. C., von Hippel P. H. Anal. Biochem. 1989; 182: 319-326

Gottschalk, S., Sparrow, J. T., Hauer, J., Mims, M. P., Leland, F. E., Woo, S. L. C., & Smith, L. C. (1996) Gene Ther. 3, 448-457.

Haensler, J. and Szoka, F. C. (1993). Bioconj. Chem. 4(5): 372-379.

Kircheis, R., Kichler, A., Wallner, G., Kursa, M., Ogris, M., Felzmann, T., Buchberger, M. and Wagner, E. (1997). Gene Therapy 4(5): 409-418.

Kren, B. T., Bandyopadhyay, P. and Steer, C. J. (1998). Nature Med. 4(3): 285-290.

Lee, R. J. and Huang, L. (1997). Crit. Rev. Ther. Drug Carrier Syst. 1997 14(2)(2): 173-206.

Nathan, A., Bolikal, D., Vyavahare, N., Zalipsky, S., Kohn, J. (1992) Macromolecules 25, 4476-4484.

Nathan, A., Zalipsky, S., Ertel, S. I., Agathos, S. N., Yarmush, M. L., Kohn, J. (1993). Bioconj. Chem. 4, 54-62.

Ogris, M., Brunner, S., Schuller, S., Kircheis, R. and Wagner, E. (1999). Gene Ther. 6(4): 595-605.

Plank, C., Mechtler, K., Szoka, F. C. and Wagner, E. (1996). Hum. Gene Ther. 7(12): 1437-1446.

Plank, C., Oberhauser, B., Mechtler, K., Koch, C., & Wagner, E. (1994) J. Biol. Chem. 269,12918-12924.

Plank, C., Tang, M., Wolfe, A. and Szoka, F. C. (1999). Hum. Gene Ther. 10(2): 319-333.

Tang, M. X., Redemann, C. T. and Szoka, F. C. (1996). Bioconj. Chem. 7(6): 703-714.

Ulbrich, K., Strohalm, J., Kopecek, J. (1985). Macromol. Chem. 187,1131-1144.

Wadhwa, M. S., Collard, W. T., Adami, R. C., McKenzie, D. L. and Rice, K. G. (1997). Bioconj. Chem. 8(1): 81-88.

Whaley, K. E. (1985). Churchill Livingstone, Edinburg, London, Melbourne and New York 1985.

Yoon, K., Colestrauss, A. and Kmiec, E. B. (1996). Proc. Natl. Acad. Sci. U.S.A. 93(5): 2071-2076.

Zalipsky, S., Gilon, C., Zilkha, A. (1984). J. Makromol. Sci.-Chem. A21, 839-845.

Zanta, M. A., Boussif, O., Adib, A., & Behr, J. P. (1997) Bioconj. Chem. 8, 839-844

Zhou, X. H. and Huang, L. (1994). Biochim. Et Biophys. Acta-Biomembr. 1189(2): 195-203.

1. A combination of a carrier and a complex containing one or more nucleic acid molecules and one or more charged copolymers of the general formula I 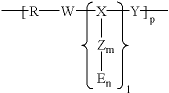
wherein R is an amphiphilic polymer or a homo- or hetero-bifunctional derivative thereof,
and wherein X
i) is an amino acid or an amino acid derivative, a peptide or a peptide derivative or a spermine or a spermidine derivative; or
ii) wherein X is 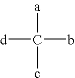
wherein
a is H or, optionally halogen- or dialkylamino-substituted, C₁-C₆ alkyl;
and wherein
b, c and d are the same or different, optionally halogen- or dialkylamino-substituted, C₁-C₆ alkylene; or
iii) wherein X is 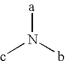
wherein
a is H or, optionally halogen or dialkylamino substituted, C₁-C₆ alkyl,
and wherein
b and c are the same or different, optionally halogen- or dialkylamino-substituted, C₁-C₆ alkylene; or
iv) wherein X
is a substituted aromatic compound with three functional groupings W₁Y₁Z₁, wherein W, Y and Z have the meanings mentioned below;
wherein
W, Y or Z have the same or different groups CO, NH, O or S or a linker grouping capable of reacting with SH, OH, NH or NH₂;
and wherein the effector molecule E is a cationic or anionic peptide or peptide derivative or a spermine or spermidine derivative or a glycosaminoglycane or a non-peptidic oligo/polycation or -anion; wherein
m and n are independently of each other 0, 1 or 2; wherein
p preferably is 3 to 20; and wherein
I is 1 to 5, preferably 1.

2. The combination according to claim 1, wherein the amphiphilic polymer of the copolymer is a polyalkylene oxide.

3. The combination according to claim 2, wherein the amphiphilic polymer of the copolymer is a polyalkylene glycol.

4. The combination according to any one of claims 1 to 3, wherein X or E in the copolymer is a charged peptide or peptide derivative.

5. The combination according to any one of claims 1 to 4, wherein a ligand for a higher eukaryotic cell is coupled to the copolymer.

6. The combination according to any one of claims 1 to 5, wherein the nucleic acid molecule(s) is (are) condensed with organic polycation or cationic lipid molecules and the complex formed thereby has one or more charged copolymers of the general formula I bound to its surface via ionic interaction.

7. The combination according to any one of claims 1 to 6, containing a therapeutically effective nucleic acid molecule.

8. The combination according to any one of claims 1 to 7, wherein the carrier consists of a biologically non-resorbable material.

9. The combination according to any one of claims 1 to 7, wherein the carrier consists of a biologically resorbable material.

10. The combination according to claim 9, wherein the biologically resorbable material is collagen.

11. The combination according to claim 10, wherein the carrier is a collagen sponge.

12. The combination according to any one of claims 1 to 7, wherein the carrier is a carrier which is obtainable by cross-linkage of a copolymer as defined in claim 1.

13. Use of a combination according to any one of claims 1 to 12 for the transfer of a nucleic acid into cells.

14. A pharmaceutical composition containing a combination according to any one of claims 1 to 12.

15. A kit containing a carrier and a copolymer or a complex as defined in claim 1.
